(19) United States
(12) Patent Application Publication
(10) Pub. No.: US 2026/0026795 A1
(43) Pub. Date: Jan. 29, 2026

(54) SURGICAL VESSEL CLOSING PRESSURE DEVICE WITH ROTATABLE BASE COVER

(71) Applicant: Catheter Precision, Inc., Fort Mill, SC (US)

(72) Inventors: Auston LOCKE, San Clemente, CA (US); David JENKINS, Flanders, NJ (US)

(21) Appl. No.: 19/347,960

(22) Filed: Oct. 2, 2025

Related U.S. Application Data
(63) Continuation-in-part of application No. 18/138,200, filed on Apr. 24, 2023.
Application No. 18/138,200 is a continuation-in-part of application No. 18/079,457, filed on Dec. 12, 2022.
Application No. 18/079,457 is a continuation-in-part of application No. 17/867,205, filed on Jul. 18, 2022.
(60) Provisional application No. 63/314,030, filed on Feb. 25, 2022.

(51) Int. Cl. A61B 17/04; A61B 17/00; A61B 90/00

(52) U.S. Cl. CPC A61B 17/04; A61B 90/08; A61B 2017/00367; A61B 2017/00429; A61B 2017/00902; A61B 2017/0496; A61B 2090/0807

(57) ABSTRACT

Various embodiments include a vessel closure pressure device that includes a pressure applying element coupled to support structure, a spindle within the support structure configured to wind up sutures fitted through a slit in the device to apply tension to the sutures, and a base cover attached to the pressure applying element having a slit partially through the bottom surface that is configured to press against the skin of a patient and to be rotated relative to the pressure applying element. In use, sutures extending from a sutured incision are slipped into the slit in the base cover and the sutured incision pressure device, and then the base cover is rotated to bring both sutures together along the centerline of the device, thereby enabling the spindle to tension both sutures evenly.

### RELATED APPLICATIONS

This application is a continuation-in-part of U.S. patent application Ser. No. 18/138,200 entitled “SURGICAL VESSEL CLOSING PRESSURE DEVICE WITH PRESSURE SENSITIVE FILM” filed Apr. 24, 2023, which is a continuation-in-part of U.S. patent application Ser. No. 18/079,457 entitled “SURGICAL VESSEL CLOSING PRESSURE DEVICE” filed Dec. 12, 2022, which is a continuation-in-part of U.S. patent application Ser. No. 17/867,205 entitled “SURGICAL VESSEL CLOSING PRESSURE DEVICE” filed Jul. 18, 2022, which claims the benefit of priority to U.S. Provisional Patent Application 63/314,030 entitled “SURGICAL VESSEL CLOSING PRESSURE DEVICE” filed Feb. 25, 2022, the entire contents of all of which are hereby incorporated by reference for all purposes.

### BACKGROUND

Currently, there are a number of medical procedures that involve inserting a catheter through the skin into a vascular vessel (e.g., a vein or artery) to gain access to various organs in the body. In such procedures, the skin is cut and the vessel is “cut down”, allowing an introducer port to be inserted. Through this port, a catheter can be inserted. These procedures encompass a wide range of minimally invasive catheter-based surgeries and procedures on major blood vessels.

A challenge faced in medical procedures including vascular vessel percutaneous access involves closing up the vessel and the incision site in the skin once the catheter and introducer are pulled out. Products have been designed to address this challenge, and can be utilized during the procedure. One such device is called “Perclose®”, and is marketed by Abbott Laboratories, Inc. (Abbott Park, Illinois, USA), while another more recently introduced device labeled for both arterial and venous access sites is called “Vascade®” marketed by Cardiva Medical, Inc. (Santa Clara, California, USA), a unit of Haemonectics Corporation (Boston, Massachusetts, USA).

While such vascular closure products assist in the closure process, one significant problem remains, which is continual bleeding at the insertion site. Such bleeding is compounded in some cases by anticoagulant therapy that some patients take as a routine therapy. To address this problem, a care giver must stay with the patient until the bleeding has stopped. Pressure is applied to the site, with some weighted bags, or most often by the care giver applying pressure with two or three fingers, for a time period of twenty minutes or more. The patient cannot be moved off the surgery table and to the recovery area during this time. As a result, the surgery room is occupied after surgery is completed, resulting in potentially less procedure throughput during any one day.

### SUMMARY

Various embodiments include devices for applying continued pressure to a vascular vessel (e.g., an artery or vein) following an intravascular procedure. Various embodiments include a pressure applying surface including a spindle for maintaining tension on suture threads extending from the sutured closure of the vascular vessel. A base cover is rotatably attached to the pressure-applying surface. The base cover includes a slit extending from one edge to the center. In use, sutures extending from a sutured incision are slipped into the slit in the base cover and the sutured incision pressure device, and then the base cover is rotated to bring both sutures together along the centerline of the device, thereby enabling the spindle to tension both sutures evenly.

In various aspects, the vessel clamping pressure device may include one or more of the following features. The base cover may include grip wings extending from opposite sides of the base cover. Each grip wing may have a grip wing contour configured to facilitate gripping by a user. The base cover may include a seat configured to receive and hold the pressure applying element therein. The base cover may include detents positioned on the upper surface of the base cover, wherein the detents are configured to resist the rotation of the base cover relative to the pressure applying element. The pressure applying element and the base cover may be sufficiently transparent to enable visualization of suture threads and skin beneath the base cover.

### BRIEF DESCRIPTION OF FIGURES

The accompanying drawings, which are incorporated herein and constitute part of this specification, illustrate exemplary embodiments, and together with the general description given above and the detailed description given below, serve to explain the features of the various embodiments.

FIG. 1A is a perspective view of a vessel clamping pressure device according to some embodiments.

FIGS. 1B and 1C are cross-sectional views of a vessel clamping vessel clamping pressure device according to some embodiments.

FIG. 1D is a cross-sectional view of the embodiment of a vessel clamping pressure device illustrated in FIG. 1C showing a portion of the device from a different viewing angle.

FIGS. 1E-1H are cross-sectional views of a vessel clamping vessel clamping pressure device showing an alternative configuration of a suture thread clamping mechanism according to some embodiments.

FIG. 2A is a perspective view of a vessel clamping pressure device according to some embodiments.

FIG. 2B is a cross-sectional view of a vessel clamping vessel clamping pressure device according to some embodiments.

FIG. 2C is a perspective view of a vessel clamping pressure device according to another embodiment.

FIGS. 2D-2I are perspective views of alternative configurations a spindle for a vessel clamping pressure device according to some embodiments.

FIG. 3 is a cross-sectional view of a vessel clamping pressure device illustrating use on a patient according to some embodiments.

FIGS. 4A-4D are perspective views of some alternative configurations of a vessel clamping pressure device according to some embodiments.

FIGS. 5A and 5B are process flow diagrams of example methods for using a vessel clamping pressure device according to some embodiments.

FIGS. 6 and 7 are perspective views of a prototype vessel clamping pressure device according to some embodiments.

FIG. 8 is a side view of a vessel clamping pressure device according to another embodiment.

FIGS. 9 and 10 are perspective views of the vessel clamping pressure device of the embodiment shown in FIG. 8.

FIG. 11 is a perspective view of a spindle component of the vessel clamping pressure device of the embodiment shown in FIG. 8.

FIGS. 12 and 13 are perspective views of a vessel clamping pressure device according to another embodiment.

FIG. 14 is a side view of the vessel clamping pressure device of the embodiment shown in FIG. 12.

FIG. 15 is a top view of the vessel clamping pressure device of the embodiment shown in FIG. 12.

FIG. 16 is an exploded view of the vessel clamping pressure device of the embodiment shown in FIG. 12.

FIGS. 17A and 17B are side views of the vessel clamping pressure device of further embodiments including a pressure-sensitive film.

FIGS. 18 and 19 are bottom views of the vessel clamping pressure device illustrated in FIG. 17 according to some embodiments.

FIG. 20 is a process flow diagram of an example method of using the vessel clamping pressure device of the embodiment shown in FIGS. 17A-19.

FIGS. 21A-21C are top, bottom, and side views, respectively, of a base cover according to some embodiments.

FIG. 22 is an exploded view of a vessel clamping pressure assembly separated from a base cover according to some embodiments.

FIG. 23A is a perspective view of an assembled version of the vessel clamping pressure assembly of FIG. 22 according to some embodiments.

FIGS. 23B-23D are top, bottom, and side views, respectively, of the vessel clamping pressure assembly of FIG. 23A according to some embodiments.

FIG. 24A is a perspective view of a vessel clamping pressure device rotated into a closed configuration relative to the base cover according to some embodiments.

FIGS. 24B and 24C are top and bottom views, respectively of the vessel clamping pressure device rotated into the closed configuration of FIG. 24A according to some embodiments.

FIG. 24D is a top view of an alternative vessel clamping pressure device rotated into a closed configuration relative to the base cover, including slits that remain partially aligned according to some embodiments.

FIGS. 25A-25C are side elevation views of an applied assembly in various configurations according to some embodiments.

FIG. 26 is a process flow diagram of an example method for applying a vessel clamping pressure device according to some embodiments.

FIG. 27 is a process flow diagram of an example method for removing a vessel clamping pressure device according to some embodiments.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims.

In overview, various embodiments include a vessel clamping pressure device that can apply tension to suture threads that have been used to close the vascular vessel, thereby bringing the pressure device in physical contact with the patient's skin sufficient to apply clamping pressure to the vessel stitches. Vessel clamping pressure devices according to various embodiments enable mechanical pressure to be provided to the closure of sutures in the vascular vessel to prompt clotting and stop bleeding from the vessel, and obviate the need for a caregiver to spend extra time with the patient following closure. In addition to providing effective pressure on the vessel sutures, the device may enable the patient to be moved out of the surgery room to the recovery area sooner than when pressure is applied by caregivers or external weights.

To close up a vascular vessel and incision site, typically a threaded suture is run through or around the vessel, most often in a figure eight type threading, with the proximal and distal portions of the thread left outside the skin to tie off. In various embodiments, rather than tying off and cutting the suture threads, the suture threads are passed through a vessel clamping pressure device, including through a suture thread tensioning mechanism that maintains the pull on the suture threads to apply a clamping pressure to the patient's skin and thus to the closure stitches in the vascular vessel.

Vessel clamping pressure devices of various embodiments may include a pressure applying surface coupled to a shaft or other support structure that includes a mechanism for maintaining tension on suture threads so that the pressure-applying surface presses against the skin of the patient. The surface area of the pressure-applying surface of the surgical device may be one to two inches square, roughly consistent with the area of a caregiver's fingers that conventionally apply pressure to the incision site after closure. The pressure-applying surface may include a passageway, such as a hole or slit, in the bottom side through which the suture threads can be passed. The pressure-applying surface may have a shape selected to match the contour of the patient's skin at the site of the incisions. For example, the pressure-applying surface may have a rounded or spherical shape to fit within a depression in the patient's body at the site of the incisions. As another example, the pressure-applying surface may have a flat, cylindrical or ellipsoidal shape to match various contours of the patient's body at the site of the incisions.

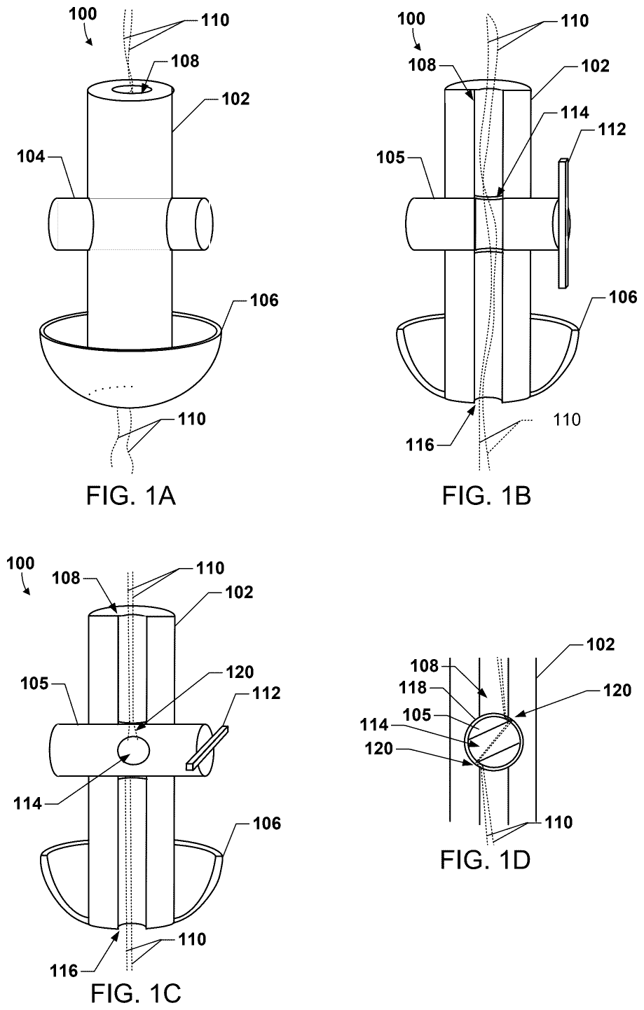

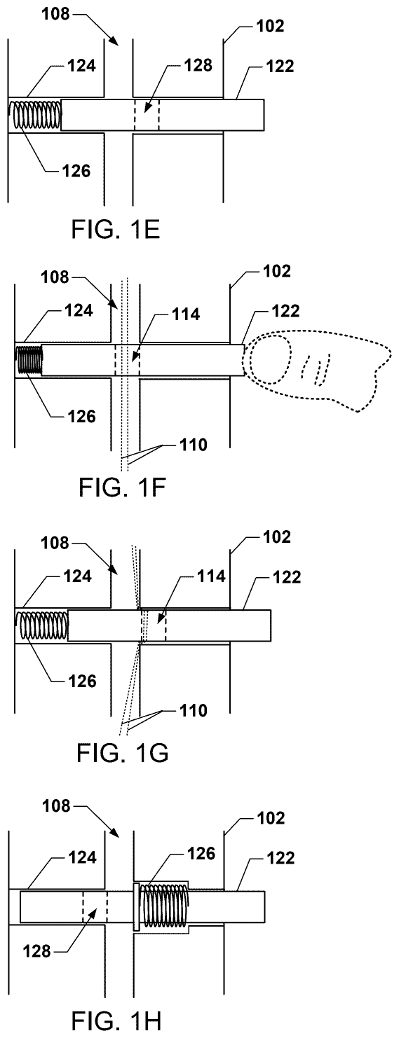

In some embodiments of vessel clamping pressure devices, the suture thread tensioning mechanism may include a suture thread clamping mechanism that a clinician can manipulate to maintain tension applied to the suture threads between the vascular vessel and the vessel clamping pressure device. In such embodiments, the suture thread tightening mechanism may include a thread gripping mechanism configured to maintain tension on suture threads after a clinician pulls the threads tight. In such embodiments, the clinician may pass the suture threads through the vessel clamping pressure device (e.g., before or after suturing the vascular vessel), pull the suture threads to tension the threads while sliding the device down the threads and against the patient's skin, and then activating the thread gripping mechanism to maintain the tension in the threads. Tensioning the suture threads and holding the threads taught via the thread gripping mechanism causes the pressure-applying surface to press against the skin of the skin of the patient, thereby applying clamping pressure to the closure of the stitches in the vascular vessel. Different thread gripping mechanisms may be used, such as a rotating clamping mechanism and a sliding clamping mechanism as described herein with reference to FIGS. 1A-1I.

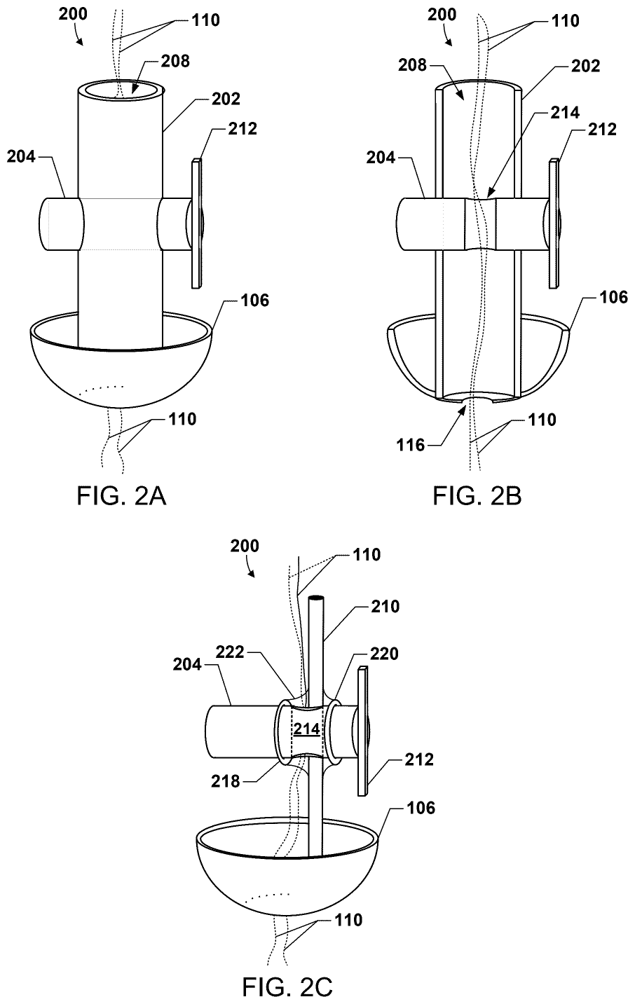

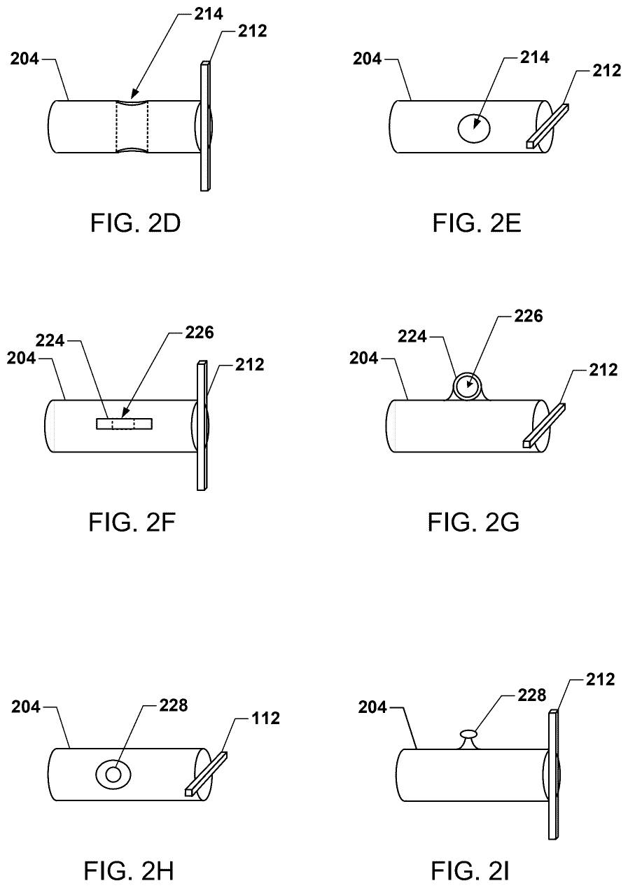

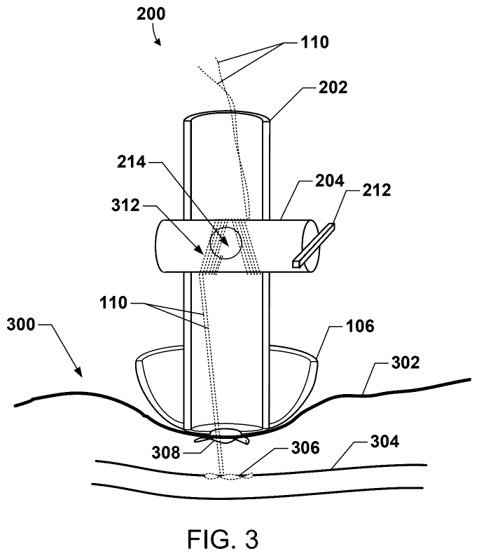

In some embodiments, the thread gripping mechanism may be in the form of a suture thread tightening mechanism coupled to the shaft that a clinician can manipulate to tension the suture threads between the vascular vessel and the vessel clamping pressure device as described herein with reference to FIGS. 2A-3. In such embodiments, the suture thread tightening mechanism may include a spindle within or coupled to a central shaft or support structure, with the spindle including a structure for receiving suture threads, such as a passageway (e.g., a hole or slit) through which suture threads may pass when the passageway is aligned with the lumen of the central shaft or support structure. The spindle may include or be coupled to a handle that can be turned to rotate the spindle, thereby binding the suture threads or rolling up slack in and applying tension to the suture threads connected to the vascular vessel. The spindle and/or the shaft or support structure may include a feature or mechanism that resists or prevents turning or unreeling of the spindle (e.g., friction, a ratchet, etc.) In some uses, a clinician may pull on the suture threads to take up any slack in the suture threads, slide the vessel clamping pressure device into contact with the patient's skin, and then rotate the spindle to bind to maintain tension on the suture threads or take up slack in the suture threads to apply further tension to the suture threads between the device and the vascular vessel. In some uses, a clinician may not need to pull on the suture threads because turning the spindle takes up slack in the threads and may apply a suitable amount of tension on the sutures. Tensioning the suture threads in this manner causes the pressure-applying surface to press against the skin of the patient, thereby applying clamping pressure to the incision site and the stitches in the vascular vessel.

In some embodiments, the pressure-applying surface may include a pressure-sensitive material or film that changes color in response to pressure, with the intensity of color depending upon the amount of pressure applied within a range. Including a color-changing pressure-sensitive material or film on the pressure-applying surface may enable a clinician to confirm when sufficient pressure is applied to support closure of the wound and/or confirm that pressure is applied evenly to the patient's skin.

The various embodiment vessel clamping pressure devices may be configured as a single-use disposable device, which may be sealed in sterile packaging before use.

FIG. 1A shows an example of a vessel clamping pressure device 100 according to some embodiments. As illustrated, a vessel clamping pressure device may include a central shaft 102 that includes a thread clamping mechanism 104 and that is coupled to a pressure applying surface 106. The central shaft 102 may include a passageway in the form of a lumen 108 through which suture threads 110 may be passed. The thread clamping mechanism 104 may be configured to enable a clinician to engage the thread clamping mechanism so as to maintain tension in the suture threads 110 leading to the stitches in a vascular vessel.

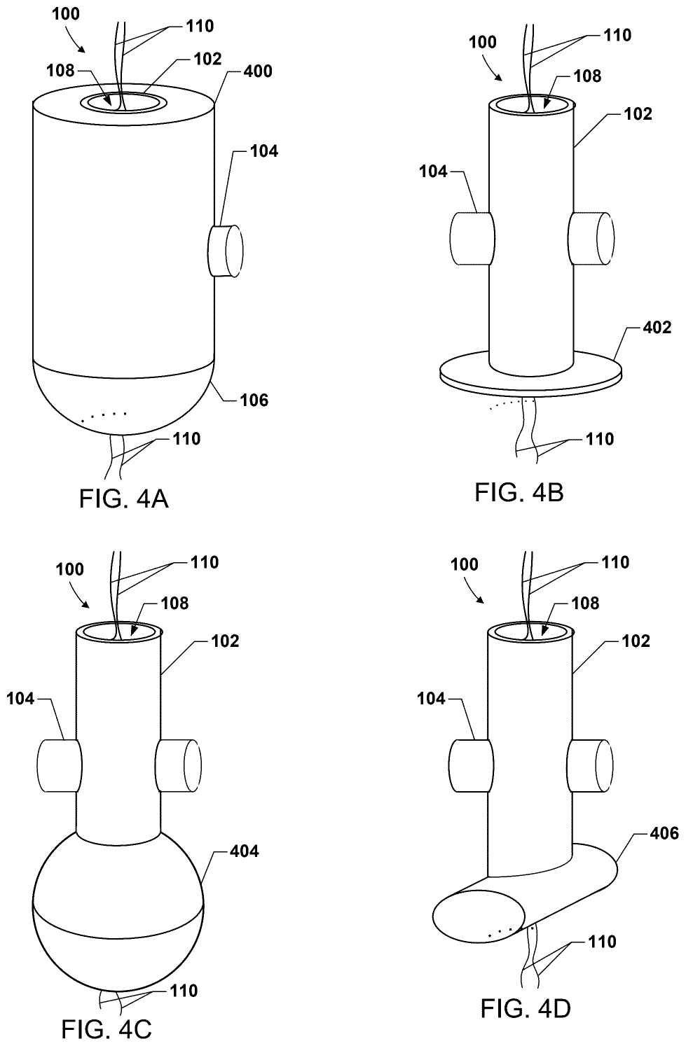

In various embodiments, the area of the pressure applying surface 106 that contacts the skin of a patient may be approximately 1 to 2 square inches. As described in more detail herein, the pressure applying surface 106 may have an external contour that is selected to match a typical contour of a patient's body at a location of an intravascular incision. For example, the pressure applying surface 106 of the embodiment illustrated in FIG. 1A is hemispherical in shape so as to fit the contours of the thigh at the location where a catheter may be inserted in the femoral artery of a patient undergoing a wide variety of non-invasive surgical and diagnostic procedures. As discussed below with reference to FIGS. 4B, 4C, and 4D, the pressure applying surface 106 may be of different shapes or contours, including flat, spherical, ellipsoidal, and irregular. In some embodiments, vessel clamping pressure devices 100 may be produced with different shaped pressure applying surfaces 106 to enable clinicians to select a suitably shaped model for use on a patient depending upon the location and surface contour of the incision site for a particular patient. In some embodiments, the vessel clamping pressure device 100 may be configured so that different shaped pressure applying surfaces 106 may be attached to a common shaft 102, enabling clinicians to select an appropriately shaped pressure applying surface depending on the location and surface contour of the incision site for a particular patient.

The shaft 102 may include a chamber for receiving the thread clamping mechanism 104. As mentioned, the thread clamping mechanism 104 may be any of be a variety of forms. FIGS. 1B-1D illustrate a non-limiting example of a thread clamping mechanism 104 in the form of a rotating spindle 105 with a passageway in the form of a hole 114, that will enable suture threads to pass through device when the spindle 105 is rotationally oriented so that the hole is aligned with the lumen 108, and jam or grip suture threads when the spindle is rotated within a complementary volume within the shaft 102 into a rotational orientation in which the hole is misaligned with the lumen. FIGS. 1E-1H illustrate a non-limiting example of a thread clamping mechanism 104 in the form of a spring-biased translating bar 122 with a passageway in the form of a hole 114 through which suture threads 110 can pass when the translating bar is depressed or pulled out so that the hole aligns with the lumen 108 and that will jam or grip suture threads against a portion of the shaft 102 when the translating bar is released so that the hole misaligns with the lumen 108.

FIGS. 1B-1D shows cross-sectional views of the embodiment of a vessel clamping pressure device 100 in which the thread clamping mechanism 104 includes a spindle 105 with a passageway in the form of a hole 114 sized to receive suture threads 110 and uses rotation to misalign the hole 114 with the lumen and press suture threads against a complementary surface in the shaft 102 and thus grip the threads so as to maintain tension on the threads. As illustrated in FIG. 2B, suture threads 110 may be passed through a passageway in the form of an opening 116 in the pressure applying surface 106, through the lumen 108 in the central shaft 102, and through the hole 114 within the spindle 105 when the spindle is rotationally oriented so that the hole aligns with the lumen. The passageway in the form of opening 116 in the pressure applying surface 106 may have a smaller diameter than the diameter of the lumen 108 of the shaft 102 so as to provide even pressure to the skin of the patient.

As illustrated in FIGS. 1C and 1D, rotating the spindle 105, rotates the hole 114 out of line (i.e., misaligned) with the lumen 108, pulling the suture threads 110 into engagement (shown with arrows 120) with a complementary surface 118 in the shaft 102. As illustrated in FIG. 1D, the complementary surface 118 may be in the form of a circular cavity with a diameter that is approximately equal to the diameter of the thread clamping mechanism 104 plus two times the diameter of the suture threads. Thus, rotating the spindle 105 causes the suture threads 110 to be pressed between the spindle 105 and the complementary surface 118, thereby providing a clamping force that maintains tension on the threads between the pressure applying surface 106 and the vascular vessel.

Passing the suture threads 110 through a passageway in the form of a hole 114 in a spindle 105 of the thread clamping mechanism 104 provides a simple mechanism for gripping the threads by rotating the spindle.

In the embodiment illustrated in FIGS. 1E-1G, the thread clamping mechanism 104 includes a spring-biased translating bar 122 that is positioned within a chamber 124 in the shaft 102 that intersects the lumen 108. The translating bar 122 may be long enough to extend beyond one side of the shaft 102. As illustrated in FIG. 1E, a spring 126 in the chamber 124 may bias the translating bar 122 so that the hole 114 in the member is normally misaligned with the lumen 108.

Referring to FIG. 1F, to pass suture threads 110 through the vessel clamping pressure device 100, such as before suturing or after closing the incision site, a clinician may press the portion of the translating bar 122 extending beyond the surface of the shaft 102, compressing the spring 126 and aligning a passageway in the form of the hole 114 in the translating bar 122 with the lumen 108 in the shaft. In this configuration, the suture threads 110 can be passed up through the opening 116 in the pressure applying surface 106, through the lumen 108 in the shaft 102, through the hole 114 within the translating bar 122 and out the top of the vessel clamping pressure device 100.

To apply pressure to the incision site, the clinician may pull on the suture threads to take up any slack, as illustrated in FIG. 1F, and press the vessel clamping pressure device 100 against the skin of the patient. Then to maintain pressure on the incision site, the clinician releases the translating bar 122, permitting the spring 126 to slide the member toward its non-depressed position, which misaligns the hole 114 in the member with the lumen 108 of the shaft, securing the suture threads 110 as illustrated in FIG. 1G. As illustrated, sliding the translating bar 122 to misalign the hole 114 in the member with the lumen 108 of the shaft 102 binds the suture threads 110 passing through the hole between the translating bar and the chamber 124.

To release the tension on the suture threads 110, such as to remove the vessel clamping pressure device 100 after a suitable period of pressure on the incision site, the clinician may again press the portion of the translating bar 122 extending beyond the surface of the shaft 102, compressing the spring 126 and aligning the hole 114 in the translating bar 122 with the lumen 108 in the shaft as illustrated in FIG. 1F.

In an alternative embodiment illustrated in FIG. 1H, the translating bar 122 may be spring loaded in a manner opposite to that illustrated in FIGS. 1E-1G such that the spring 126 is oriented to resist a pulling action on the translating bar. In this embodiment, a clinician may pull (rather than push as illustrated in FIG. 1F) the translating bar to align a passageway in the form of a hole 128 with the lumen 108 in the shaft 102 for passing suture threads through the vessel clamping pressure device 100. In this embodiment, pulling on the translating bar 122 compresses the spring 124, which provides force to return the translating bar to the illustrated position in which the hole 128 is misaligned with the lumen 108, thus binding the suture threads 110 passing through the passageway in the form of a hole 128 between the translating bar and the chamber 124 in a manner similar to the illustration in FIG. 1G.

In the embodiment illustrated in FIG. 1H, a clinician may pull on the translating bar 122 to pass suture threads through the vessel clamping pressure device 100 and press the device against the patient's skin while tensioning the threads with one hand, and then release the translating bar to maintain the tension on the suture threads and thus the clamping force on the sutured incisions. To remove the vessel clamping pressure device 100, the clinician may pull on the translating bar 122 to align the hole 114 with the lumen 108, enabling the suture threads 110 to pass through the hole, releasing the tension and enabling the threads to be removed from the device.

In various embodiments, the length of the shaft 102 above the spindle 104 or translating bar 122 may vary from the relative amount illustrated in the figures. For example, the shaft 102 may end just above the spindle 104 or the translating bar 122. Further, in some embodiments, the length of the shaft 102 between the spindle 104 or translating bar 122 and the pressure applying surface 106 may vary from the relative amount illustrated in the figures. For example, the distance between the spindle 104 or translating bar 122 and the pressure applying surface 106 may be just long enough to permit actuation of the thread clamping mechanism 104.

FIGS. 2A-2I shows an example of a vessel clamping pressure device 200 according to some further embodiments. As illustrated, a vessel clamping pressure device 200 according to such embodiments may include a central shaft 202 that includes a rotatable spindle 204 and that is coupled to the pressure applying surface 106. Similar to the embodiments illustrated in FIGS. 1A-1G, the area of the pressure applying surface 106 that contacts the skin of a patient may be approximately 1 to 2 square inches and have an external contour that is selected to match a typical contour of a patient's body at a location of an intravascular incision.

The central shaft 202 may include a passageway in the form of a lumen 208 through which suture threads 110 may be passed. The spindle 204 may include a handle 212 or other structure that enables a clinician to rotate the spindle so as to tension (or maintain tension on) suture threads 110 leading to the stitches in a vascular vessel. The shaft 202 may include an opening for receiving the spindle 204 and allowing the spindle to be rotated about its long axis. The shaft 202 and/or the spindle 204 may further include a mechanism (not shown separately) for limiting unwinding of the spindle after the suture threads have been tightened. Any of a variety of unwinding limiting mechanism may be used, including friction between the spindle and the shaft, a ratchet mechanism that permits rotation in one direction but not the other, a tooth and gear interface that permits rotation of the spindle when pushed in but resists rotation when released, etc.

FIG. 2B shows a cross-sectional view of the embodiment of a vessel clamping pressure device 200 shown in FIG. 2A. As illustrated, suture threads 110 may be passed through a passageway in the form of an opening 116 in the pressure applying surface 106, through the lumen 208 in the central shaft 202, and through a hole 214 within the spindle 204. The passageway or opening 116 in the pressure applying surface 106 may have a smaller diameter than the diameter of the lumen 208 of the shaft 202 so as to provide even pressure to the skin of the patient.

Passing the suture threads 110 through the hole 214 in the spindle 204 provides a simple mechanism for coupling the threads to the spindle so that when the spindle is rotated, the suture threads between the spindle and the vascular vessel are tightened, as illustrated in FIG. 3.

In various embodiments, the length of the shaft 202 above the spindle 204 may vary from the relative amount illustrated in the figures. For example, the shaft 202 may end just above the spindle 204. Further, in some embodiments, the length of the shaft 202 between the spindle 204 and the pressure applying surface 106 may vary from the relative amount illustrated in the figures. For example, the distance between the spindle 204 and the pressure applying surface 106 may be just long enough to accommodate windings of the suture threads 110 about the spindle 204.

In some embodiments, the shaft 202 may not be tubular as illustrated in the drawings, and instead may be any of a variety of structures that support the spindle 204 and connect to the pressure applying surface 106. For example, as illustrated in FIG. 2C, the shaft 210 may be a solid rod coupled to one or more bearings 218, 220 via a support structure 222, with the bearings 218, 220 configured to provide rotational support for the spindle 204. In such an embodiment, the suture threads 110 may pass through a passageway in the form of the opening 116 in the pressure applying service 106 and through a passageway in the form of the hole 214 in the spindle 204 without passing through a lumen in the shaft 210. In some embodiments, the shaft 210 may be shorter relative to the pressure apply surface 106 than illustrated, and configured to support the bearings 218, 220 on the pressure apply surface 106 to enable rotation of the spindle 204.

The spindle 204 may include a variety of features for securing the suture threads 110 for winding. Three non-limiting alternatives are illustrated in FIGS. 2D-2I.

FIGS. 2D and 2E show an embodiment of a spindle 204 including a passageway in the form of a hole 214 through which suture threads 110 may be passed. In such embodiments, the hole 214 may have a diameter sufficient to receive the suture threads 110. In some embodiments, the hole 214 may have a diameter similar to or the same as the diameter of the lumen 208 in the shaft 202. Rotating the handle 212 on the spindle 204 (e.g., as shown in FIG. 2B) then causes the suture threads 110 to be wound about the spindle as illustrated in FIG. 3.

FIGS. 2F and 2G show an embodiment of a spindle 204 including a ring 224 or similar structure having a passageway in the form of a hole 226 through which suture threads 110 may be passed. In this embodiment, suture threads may be passed through the hole 226 when the spindle 204 is turned so that the ring 224 is oriented approximately perpendicular to the long axis of the shaft 202, which orients the axis of the hole 226 approximately parallel to the long axis of the shaft 202. Similar to the embodiments illustrated in FIG. 2A-2E, turning the spindle 204 will wrap suture threads 110 around the spindle in a manner similar to the example illustrated in FIG. 3.

FIGS. 2H and 2I show an embodiment of a spindle 204 having a structure, such as a knob 228 as illustrated, a hook, or similar structure, that may catch or secure suture threads 110 so that the threads will be wound around the spindle when it is rotated. For example, in an embodiment of a vessel clamping pressure device 200 in which the spindle 204 includes a knob 228, the suture threads 110 may be wound around the knob to secure the threads to the spindle before winding the spindle. Such an embodiment of the spindle 204 may be useful in a vessel clamping pressure device 200 embodiments with a solid shaft 210 as illustrated in FIG. 2C in which the knob 228 is not hidden from view or access within a lumen 208 of the shaft. With suture threads 110 wound around or otherwise connected to the knob 228 (or similar structure), turning the spindle 204 will wrap suture threads 110 around the spindle in a manner similar to the example illustrated in FIG. 3.

While not illustrated in FIGS. 1A-2I, a tool such as a straight needle may be used to push or pull the suture threads 110 through the passageway in the vessel clamping pressure device 100, 200, including through a hole 114 in the spindle 105, 204 or translating bar 122, which may be hidden from view depending upon the materials used in the central shaft 102. As another alternative, the suture threads 110 may be threaded through the passageway in the vessel clamping pressure device 100, 200 before (or in preparation for) the suture threads are used to stitch or otherwise close the incision in the vascular vessel.

FIG. 3 shows a cross-sectional view of an embodiment of a vessel clamping pressure device 200 in which the spindle 204 has been rotated a few turns, winding suture threads 110 around the spindle sufficient to tighten the suture threads 110 between the spindle 204 and the vascular vessel 304. As illustrated in FIG. 3, slack in the suture threads 110 between the spindle 204 and the vascular vessel 304 is taken up in windings 312 on the spindle. Tightening the suture threads 110 draws the vessel clamping pressure device 200 toward the vascular vessel 304, which causes the pressure applying surface 106 to press against the skin 302 of a patient 300 at the incision site closure 308. With suture threads 110 extending from the vascular vessel 304, such as from sutures 306 in the vessel, a clamping force may be induced between the vascular vessel 304 and the pressure applying surface 106 (and the patient's skin 302) that can stop or minimize bleeding and accelerate clotting at the site of the incision in the vessel without the need for a clinician to apply external pressure to the incision site closure.

A vessel clamping pressure device 100, 200 according to various embodiments may vary in form and structure from that illustrated in FIGS. 1A-3, some non-limiting examples of which are illustrated in FIGS. 4A-4D.

For example, as illustrated in FIG. 4A, a vessel clamping pressure device 100, 200 may include a case or shell 400 that encompasses the central shaft 102, 202 fitting against the pressure applying surface 106. In some embodiments, the central shaft 102, 202 may have an outer diameter (or outer surface contour) that matches the pressure applying surface 106, in which case the central shaft 102, 202 would have the shape of the shell 400. By hiding surface contours that could harbor bacteria, such a configuration of a vessel clamping pressure device 100, 200 may facilitate sterilizing the device before packaging, as well as after use for devices configured for reuse (i.e., not single-use disposable models).

As noted above, the pressure applying surface 106 may have a variety of shapes and contours that may be selected to match the skin of the patient at the site of the incisions, some non-limiting examples of which are illustrated in FIGS. 4B-4D. As illustrated in FIG. 4B, the pressure applying surface 402 may be flat or nearly flat, such as a disk having an area of approximately one to two square inches. As illustrated in FIG. 4C, the pressure applying surface may be a solid shape, such as a sphere 404, with a radius selected so that the portion of the solid shape that contacts a patient's skin has a surface area of approximately one to two square inches. As illustrated in FIG. 4D, the pressure applying surface may be a complex shape, such as an elongated ellipsoid 406, with major, minor, and longitudinal axes selected so that the portion of the shape that contacts a patient's skin has a surface area of approximately one to two square inches.

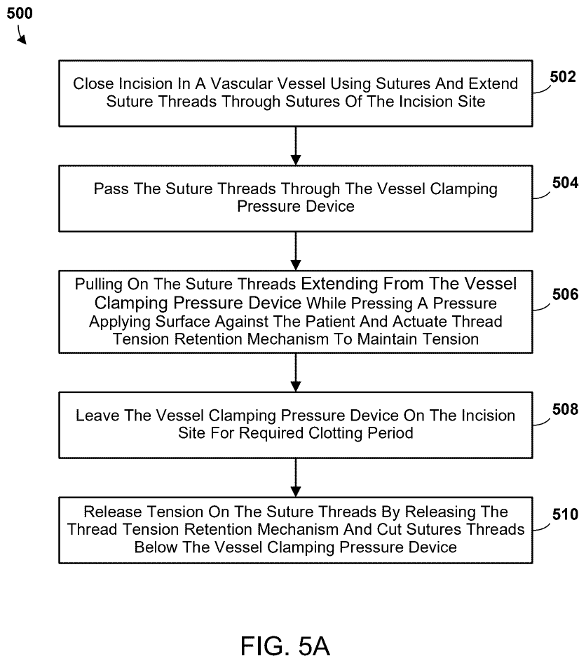

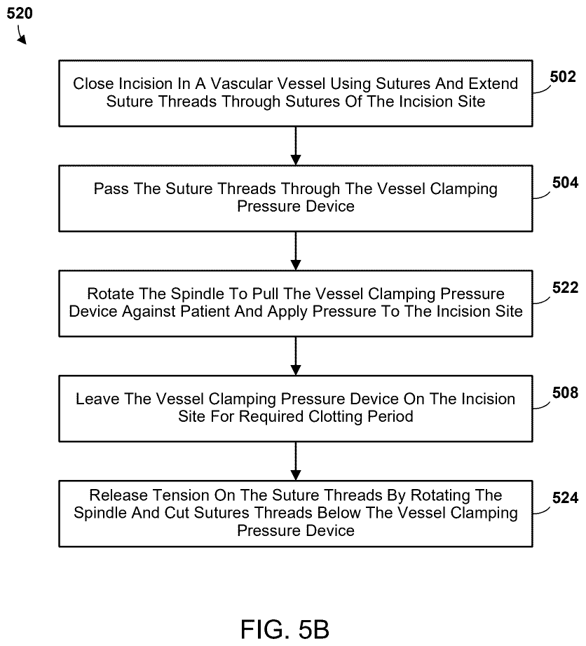

FIG. 5A is a process flow diagram illustrating a method 500 for using a vessel clamping pressure device 100 according to some embodiments. With reference to FIGS. 1A-1I, the method 500 may be performed as part of the closure of a vascular vessel following a catheterization procedure.

In block 502, a clinician may close the incision in a vascular vessel using sutures, and extend the suture threads (e.g., 110) through sutures of the entrance incision, which is then sutured close.

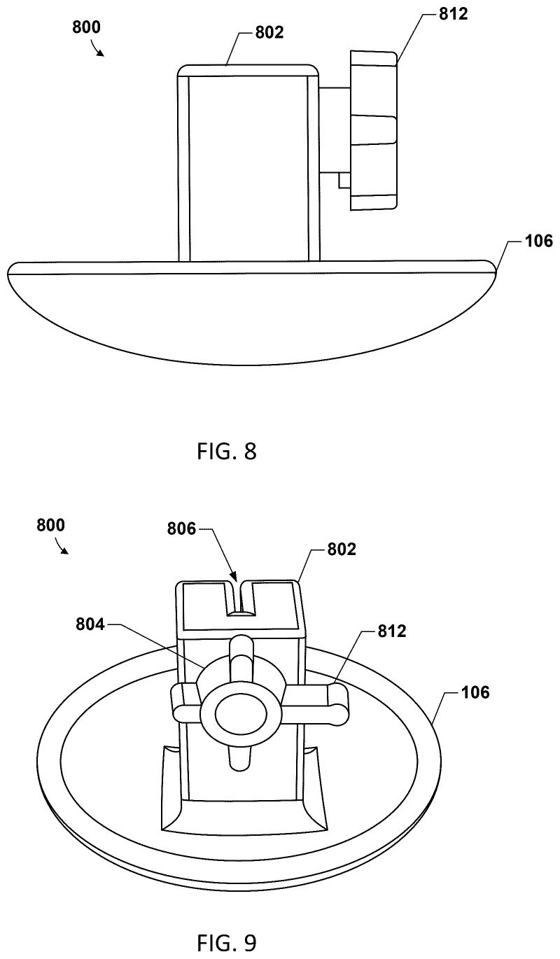

In block 504, a clinician may pass the suture threads (e.g., 110) through a passageway through the vessel clamping pressure device 100. As described herein, this operation may involve passing the suture threads through a hole 116 in the pressure applying surface (e.g., 106, 402, 404, 406), through a hole (e.g., 114, 228) in/on the thread clamping mechanism 104, and out the top of the vessel clamping pressure device 100. As described with reference to FIGS. 8-11, this operation may involve slipping the suture threads into slits that provide a passageway through the vessel clamping pressure device 800.

In block 506, a clinician may pull on the suture threads extending from the vessel clamping pressure device to tension the threads while pressing the vessel clamping pressure device against the patient to apply pressure to the incision site, and then actuate the thread tension retention mechanism (e.g., 104, 105, 122) to maintain the tension in the threads and thus the pressure against the incision site. As discussed herein, in some embodiments, actuating the thread tension retention mechanism may involve rotating a spindle (e.g., 105) with a hole (e.g., 114) through which the suture threads (e.g., 110) pass so as to grip the threads between the spindle and a corresponding surface in a shaft (e.g., 102) of the vessel clamping pressure device. Also as discussed herein, in some embodiments, tensioning the threads while pressing the vessel clamping pressure device against the patient in block 506 may include pressing or pulling on a translating bar (e.g., 122) to align a hole (e.g., 144) in the bar with a lumen (e.g., 108) in the shaft (e.g., 102) to enable the suture threads to pass through the hole, and actuating the thread tension retention mechanism may include releasing the translating bar to misalign the hole with the lumen to grip the threads between the translating bar and a chamber (e.g., 124) in the shaft of the vessel clamping pressure device.

In block 508, a clinician may leave the vessel clamping pressure device on the incision site for a required clotting period. At this point, the patient may be moved out of surgery, such as to recovery.

In block 510, after a sufficient period of time, a clinician may release tension on the suture threads by actuating the thread tension retention mechanism and cut the suture threads below the vessel clamping pressure device. As discussed herein, in some embodiments, actuating the thread tension retention mechanism to release tension on the suture threads may involve rotating a spindle (e.g., 105) to align a passageway in the form of a hole (e.g., 114) in the spindle with the lumen in the shaft, allowing the threads to pass through the hole. Also as discussed herein, in some embodiments, actuating the thread tension retention mechanism to release tension on the suture threads may involve pressing or pulling on a translating bar (e.g., 122) to align a passageway in the form of a hole (e.g., 144) in the bar with the lumen in the shaft to enable the suture threads to pass through the hole.

After removal from the client, vessel clamping pressure devices configured as single-use disposable items may be disposed of.

FIG. 5B is a process flow diagram illustrating a method 520 for using a vessel clamping pressure device 200 according to some embodiments. With reference to FIGS. 2A-5B, the method 500 may be performed as part of the closure of a vascular vessel following a catheterization procedure.

In block 502, a clinician may close the incision in a vascular vessel using sutures, and extend the suture threads (e.g., 110) through sutures of the entrance incision, which is then sutured close.

In block 504, a clinician may pass the suture threads (e.g., 110) through a passageway in the vessel clamping pressure device 200. As described above, this operation may involve passing the suture threads through a hole 116 in the pressure applying surface (e.g., 106, 402, 404, 406), through a hole (e.g., 214, 228) in/on a spindle 204 (or engage a structure such as a knob 228 or hook on the spindle), and out the top of the vessel clamping pressure device 200. As described with reference to FIGS. 8-11, this operation may involve slipping the suture threads into slits that provide a passageway through the vessel clamping pressure device 800.

In block 522, a clinician may rotate the spindle to tension the suture threads between the spindle 204 and the vascular vessel, thereby pulling the pressure applying surface of the vessel clamping pressure device against the patient and applying pressure to the incision site. This operation may include engaging a mechanism to prevent the spindle from unwinding, such as engaging a ratchet or tightening a tension nut on the spindle.

In block 508, a clinician may leave the vessel clamping pressure device on the incision site for a required clotting period. At this point, the patient may be moved out of surgery, such as to recovery.

In block 524, after a sufficient period of time, a clinician may release tension on the suture threads by rotating the spindle to unwind threads and cut the suture threads below the vessel clamping pressure device.

After removal from the client, vessel clamping pressure devices configured as single-use disposable items may be disposed of.

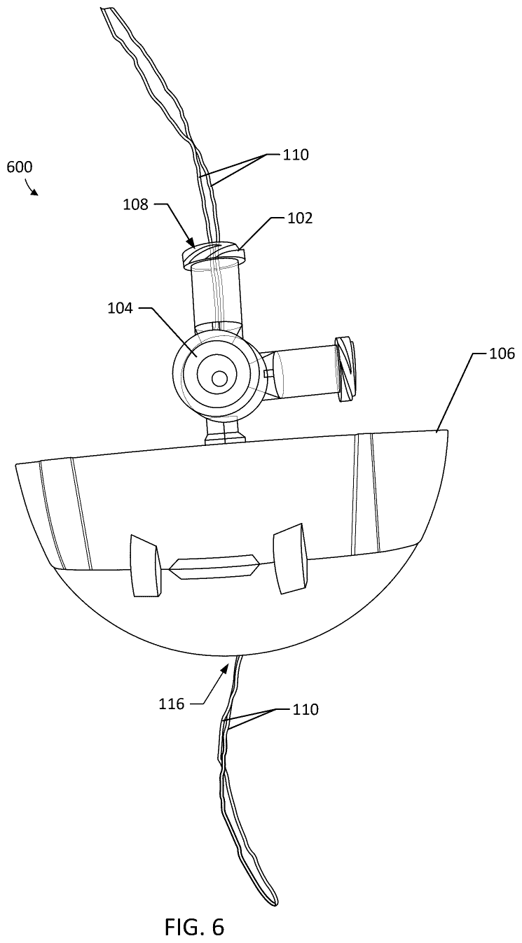

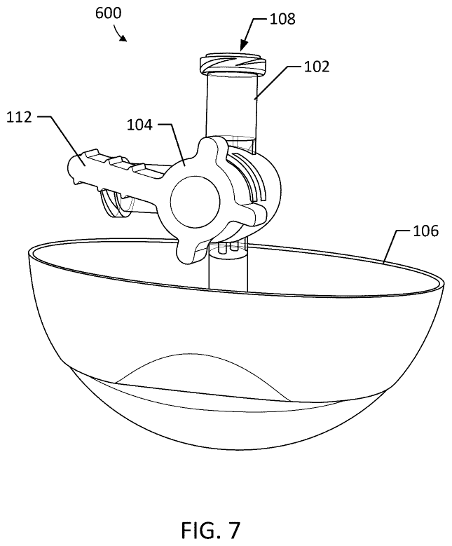

FIGS. 6 and 7 are illustrations of a prototype vessel clamping pressure device 600 according to a non-limiting embodiment. In this prototype, a stopcock is used for the shaft 102 and spindle 104. FIG. 6 shows one side of the prototype clamping pressure device 600 with suture threads 110 passing through the lumen 108 within the valve body, through the spindle 104 (i.e., through the hole in the valve plug) and through an exit hole 116 in the hemispherical pressure applying surface 106. As described herein, when the spindle 104 (i.e., the valve plug) is rotated, the suture threads 110 will wrap around the spindle, thus tightening (or maintaining tension on) the suture threads between the pressure applying surface 106 and the patient. FIG. 7 shows the other side of the prototype clamping pressure device 600 from an angle that shows the handle 112 of the spindle 104 (i.e., the valve plug).

FIGS. 8-11 are illustrations of another embodiment of a vessel clamping pressure device 800. Like other embodiments, the vessel clamping pressure device 800 may include a central shaft 802 coupled to the pressure applying surface 106. In the embodiment illustrated in FIGS. 8-11, the central shaft 802 may have a rectangular cross-section as illustrated, or other configuration. In some embodiments, the central shaft 802 may be glued, fused, or otherwise firmly coupled to the pressure applying surface 106. In some embodiments, the central shaft 802 and the pressure applying surface 106 may be manufactured as a single structure, such as in a single mold or using additive manufacturing technologies (known as 3D printing).

A spindle 804 may fit into the central shaft 802 and be configured to turn within the shaft. The spindle 804 may include a handle 812 to facilitate turning the spindle 804. As illustrated in FIG. 11, the spindle 804 may include an interior portion 810 that fits within the central shaft 802 and includes slits 814 that form a spindle passageway through which sutures may be passed.

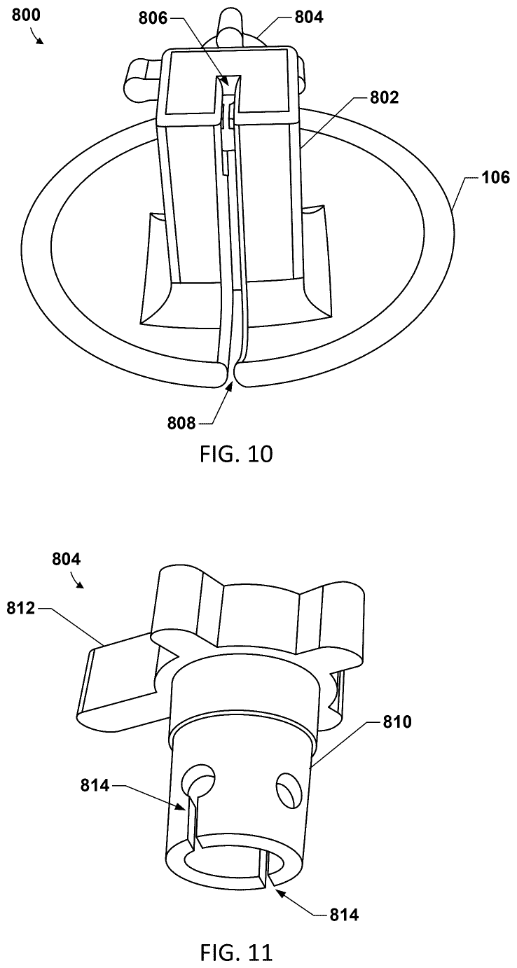

As illustrated in FIGS. 9 and 10, the central shaft 802 may include a passageway in the form of a slit 806 that matches up with a passageway in the form of a slit 808 in the pressure applying surface 106. The passageway slits 806, 808 in the central shaft 802 and the pressure applying surface 106 provide a passageway for sutures through the vessel clamping pressure device 800. The passageway in the form of a slit 808 in the central shaft 802 may be sized to match up with the spindle passageway in the form of slits 814 in the interior portion 810 of the spindle 804 when the spindle is positioned within the central shaft. The passageway slits 806, 808, and 814 in the central shaft 802, pressure applying surface 106, and spindle interior portion 810 enable the vessel clamping pressure device 800 to be attached to sutures without having to thread the sutures through an interior passage as in other embodiments described herein.

To connect the vessel clamping pressure device 800 to sutures, a clinician may turn the spindle 804 to align the spindle passageway slits 814 in the interior portion 810 with the slits 806, 808 in the central shaft 802 and pressure applying surface 106, and then slip the sutures into the passageway through the vessel clamping pressure device 800 formed by the slits. This passes the sutures through the spindle 804. A clinician may press the vessel clamping pressure device 800 against the incision site while tensioning the sutures above the device, and then rotate the spindle 804, which binds the sutures between the interior portion 810 and a corresponding surface on the central shaft 802. Binding the sutures in this manner maintains the tension in the sutures between the vessel clamping pressure device 800 and the suture site, thereby maintaining the pressure applied to the incision site by the pressure applying surface 106. To release the pressure on the incision site, a clinician may rotate the spindle 804 to realign the slits 814 in the interior portion 810 with the slits 806, 808 in the central shaft 802 and pressure applying surface 106, which releases the sutures and enables the vessel clamping pressure device 800 to be removed from the sutures.

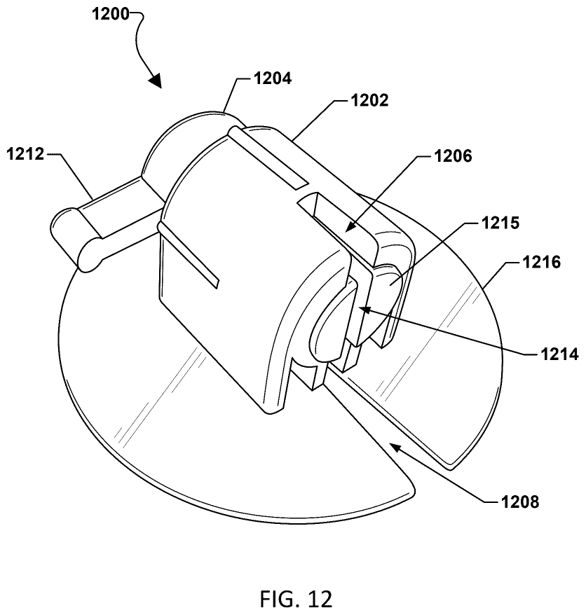

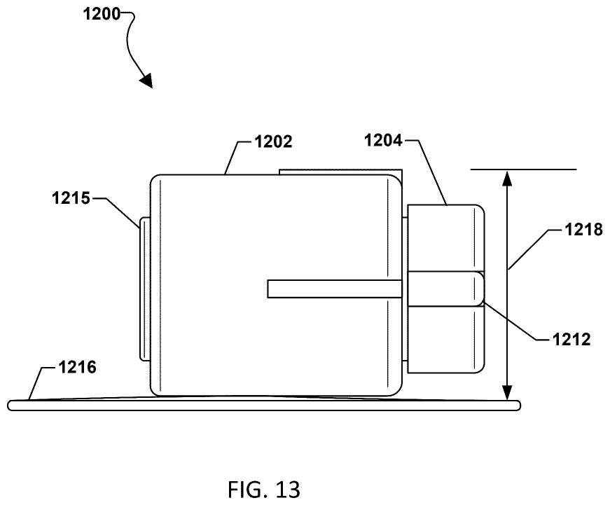

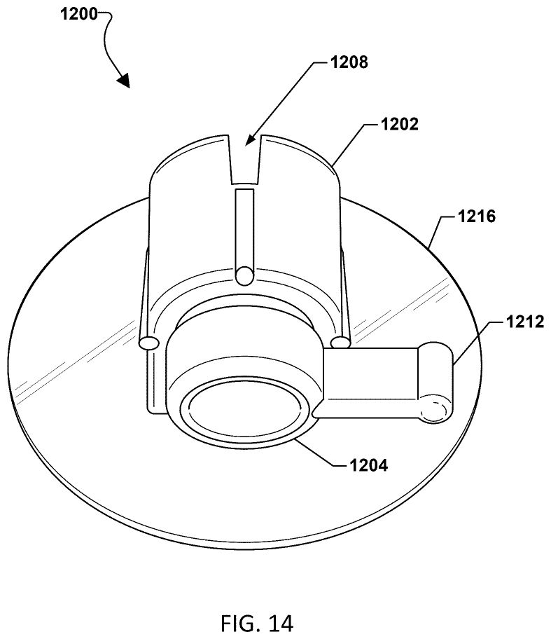

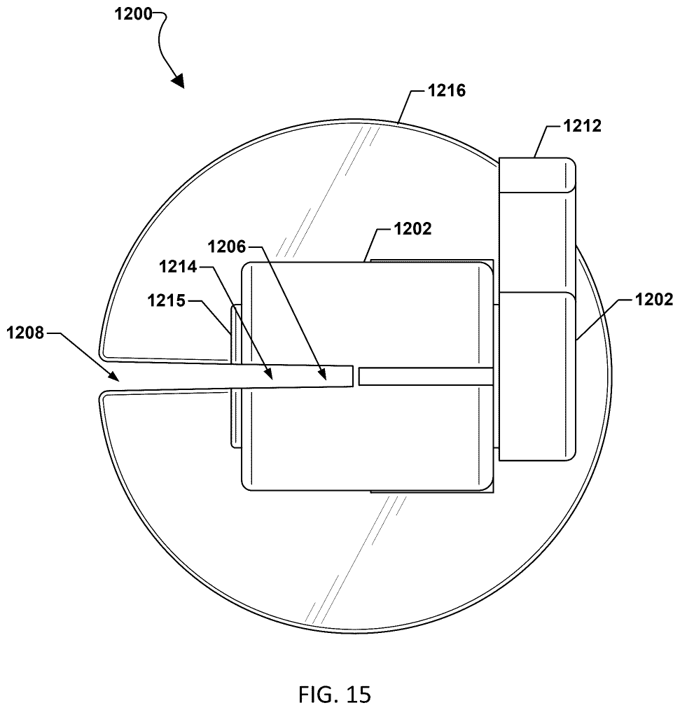

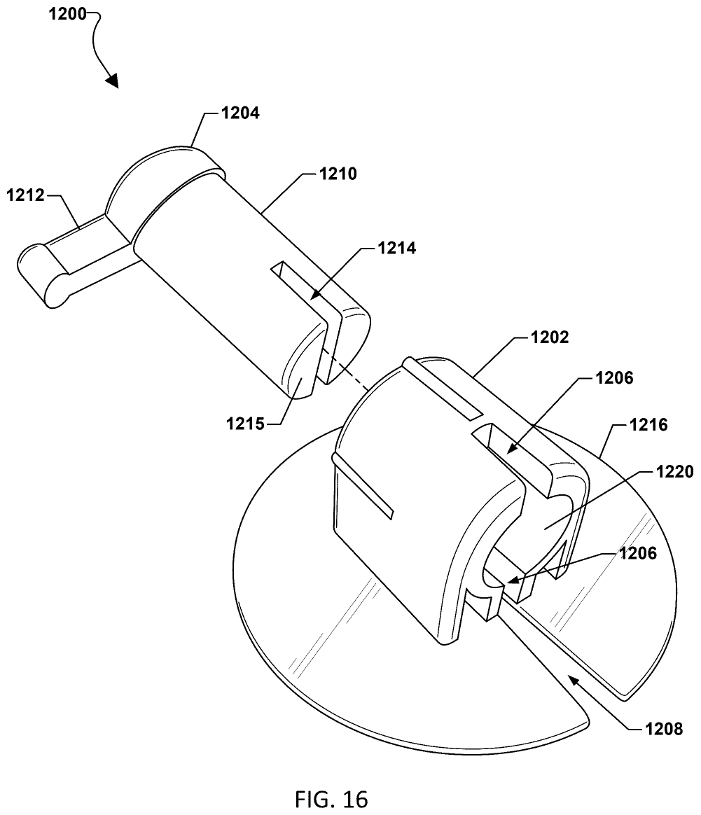

FIGS. 12-16 are illustrations of another embodiment of a vessel clamping pressure device 1200. Like other embodiments, the vessel clamping pressure device 1200 may include a central shaft, referred to in this embodiment as a support structure 1202 coupled to a pressure applying element, referred to in various embodiments as the pressure applying surface 1216. Referring to FIGS. 12-16, in this embodiment, the support structure 1202 may be of a reduced height 1218 compared to other embodiments illustrated herein, thereby reducing the overall height dimension (i.e., from the pressure applying surface 1216 to a top of the support structure 1202). Minimizing the height 1218 may facilitate including the vessel clamping pressure device 1200 within bandages.

The support structure 1202 may include a through-hole 1220 (see FIG. 16) sized to receive a spindle 1204. In some embodiments, the support structure 1202 may have a rectangular cross section as illustrated, or another configuration. In some embodiments, the support structure 1202 may be glued, fused or otherwise firmly coupled to the pressure applying surface 1216. In some embodiments, the support structure 1202 and the pressure applying surface 1216 may be manufactured as a single structure, such as in a single mold or using additive manufacturing technologies (known as 3D printing).

The pressure applying surface 1216 is configured to be applied to the skin of a patient at the site of a wound closure. In some embodiments, the pressure applying surface 1216 may be flat as illustrated or curved as shown in other figures. In some embodiments, the pressure applying surface 1216 may be transparent (as illustrated) or translucent, such as clear plastic, which may be sufficiently transparent to aid a clinician in applying the device to a patient and/or assessing the healing state of the wound. In some embodiments, the pressure applying surface 1216 may be circular as illustrated or another shape, such as a shape configured to better match a location on the patient where the device may be attached.

The spindle 1204 may fit into the through-hole 1220 of the support structure 1202 and be configured to turn within the support structure. The spindle 1204 may include a front face 1215 at one end and a handle 1212 at the other end to facilitate turning the spindle 1204. As illustrated in FIG. 16, spindle 1204 may include an interior portion 1210 that fits into the through-hole 1220 in the support structure 1202 and the front face 1215 includes a slit 1214 that forms a spindle passageway through which sutures may be passed as described herein. In particular, the slit 1214 is configured to allow a broadside insertion of a longitudinal extend of one or more sutures therein. In some embodiments, the spindle 1204 and the through-hole 1220 in the support structure 1202 may be configured so that the spindle 1204 can be turned through at least 180 degrees. In some embodiments, the spindle 1204 and the through-hole 1220 in the support structure 1202 may be configured so that the spindle 1204 can be turned through approximately 90 degrees. Turning the spindle 1204 from the orientation illustrated in the figures by 90 degrees (i.e., turning the handle 1212 vertically) rotates the slit 1214, which will engage the sutures, thereby maintaining tension on the sutures as described herein.

As illustrated in FIGS. 12, 15, and 16, the support structure 1202 may include a passageway in the form of a slit 1206 that matches up with a passageway in the form of a slit 1208 in the pressure applying surface 1216. The slits 1206, 1208 in the support structure 1202 and the pressure applying surface 1216 provide a passageway for sutures through the vessel clamping pressure device 1200. The slit 1208 in the support structure 1202 may be sized to match up with the slit 1214 in the interior portion 1210 of the spindle 1204 when the spindle is positioned within the support structure. The passageway slits 1206, 1208, and 1214 in the support structure 1202, pressure applying surface 1216, and spindle interior portion 1210 enable the vessel clamping pressure device 1200 to be attached to sutures without having to thread the sutures through an interior passage, as in other embodiments described herein. In some embodiments, the support structure 1202 may be transparent or translucent, such as clear plastic, which may aid a clinician in positioning the vessel clamping pressure device 1200 over the wound and slipping the suture into the slit 1214 in the spindle 1208.

To connect the vessel clamping pressure device 1200 to sutures, a clinician may turn the spindle 1204 to align the spindle passageway slits 1214 in the interior portion 1210 with the slits 1206, 1208 in the support structure 1202 and pressure applying surface 1216. The clinician may then align the center of the vessel clamping pressure device 1200 with the wound site, which has sutures protruding therefrom, such as by looking through the transparent pressure applying surface 1216. Once positioned appropriately, the clinician may slip a broadside insertion of the protruding sutures into the passageway through the vessel clamping pressure device 1200 formed by the slits 1206, 1208, 1216, thereby passing the sutures through the spindle 1204. A clinician may press the vessel clamping pressure device 1200 against the incision site while tensioning the sutures above the device, and then rotate the spindle 1204, which binds the sutures between the interior portion 1210 and a corresponding surface of the through-hole in the support structure 1202. Binding the sutures in this manner maintains the tension in the sutures between the vessel clamping pressure device 1200 and the suture site, thereby maintaining the pressure applied to the incision site by the pressure applying surface 1216. With the reduced height 1218 of this embodiment, the clinician may apply bandages over the device 1200 to protect the wound site. To release the pressure on the incision site, a clinician may rotate the spindle 1204 to realign the slits 1214 in the interior portion 1210 with the slits 1206, 1208 in the support structure 1202 and the pressure applying surface 1216, which releases the sutures and enables the vessel clamping pressure device 1200 to be removed from the sutures.

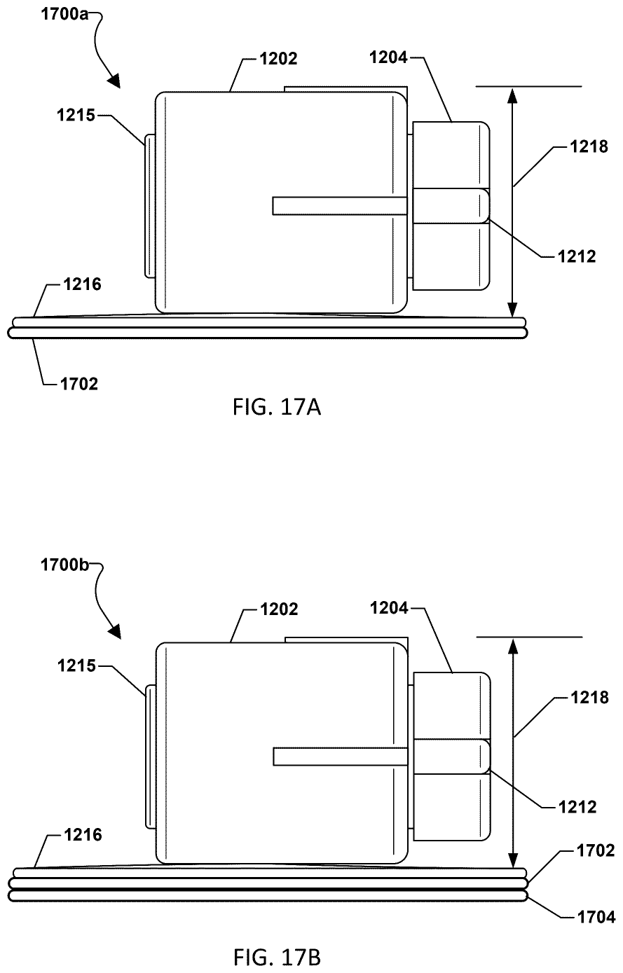

FIGS. 17-19 are illustrations of another embodiment of a vessel clamping pressure device 1700 that includes pressure-sensitive film or films 1702, 1802-1810, 1902, such as manufactured by Fujifilm. Pressure-sensitive films change color when pressure is applied to the film. Including pressure-sensitive films on the pressure applying surface 1216 may enable the clinician to confirm that sufficient pressure is being applied and/or being applied evenly to the patient by the vessel clamping pressure device 1700.

Any of a variety of pressure-sensitive films, tapes, or sheets may be used in various embodiments. As an enabling but non-limiting example, Fujifilm manufactures a pressure sensitive film that is suitable for some embodiments. A type of pressure-sensitive film manufactured by Fujifilm is a two-sheet system that made of two films, A-film and C-film, both of which have a base material made of PET (Polyethylene Terephthalate). The A-film is coated with a color-forming material in the form of microcapsules. The C-film is coated with a color-developing material. When pressure is applied to the two films, the microcapsules on the A-film rupture, transferring the color-forming material to the color-developing material on the C-film. The interaction between the two materials generates a red color, thereby creating a visual representation of the pressure exerted on the film. Pressure-sensitive films made by Fujifilm are distributed in the U.S. by Pressure Metrics LLC as “Prescal” and “Prescale Sheet.”

The Fujifilm pressure sensitive films (the A-film and the C-film) are made of PET, which is a colorless thermoplastic polymer resin in the polyester family that is transparent in its natural state. PET is generally considered safe for contact with human skin, as it is a stable and inert material, and is often used in various medical applications.

As illustrated in FIG. 17A, the pressure-sensitive film 1702 may be positioned on the patient-facing surface of the pressure applying surface 1216 and configured to render the surface more comfortable to the patient. Pressure-sensitive films made of PET, such as the films manufactured by Fujifilm, may be applied directly to the skin of the patient due to the inert nature of the material.

Some pressure-sensitive films suitable for use in some embodiments may not be suitable for being placed directly on the skin of the patient. To accommodate such pressure-sensitive materials, some embodiments, an example of which is illustrated in FIG. 17B, may include a cover sheet 1704 of a material that is safe to apply to a patient's skin, such as thermoplastic, PET, glass, or similar materials. In such embodiments, as the pressure applying surface (which is the exterior surface of the cover sheet 1704) is pressed against the patient while tension is applied to the sutures, the pressure-sensitive film 1702 (or similar structure) is squeezed between the cover sheet 1704 and the pressure applying surface 1216, leading to a color change.

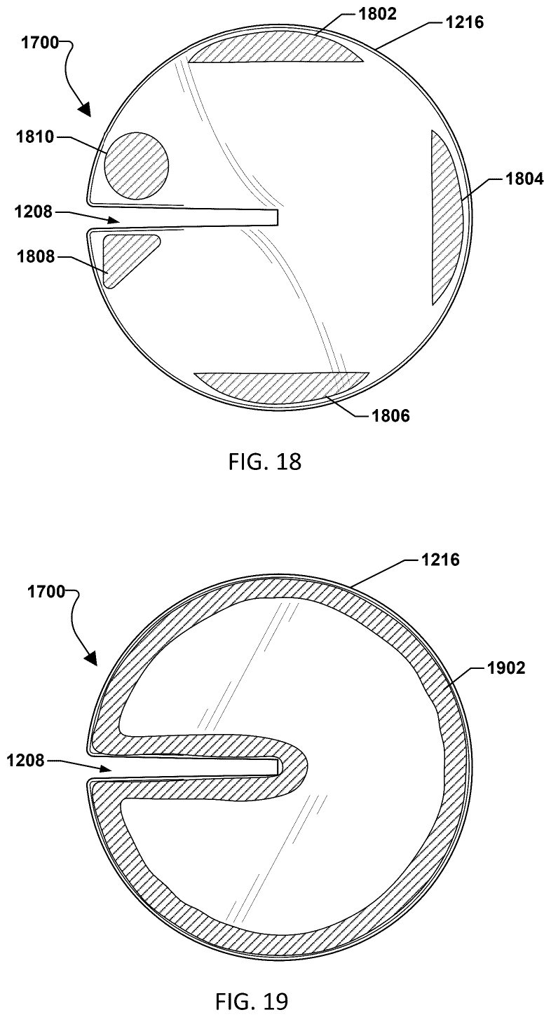

In some embodiments, the pressure-sensitive film may be applied in different shaped patches 1802-1810 and on different portions of the patient-facing surface of the pressure applying surface 1216, as illustrated in FIG. 18. For example, pressure-sensitive film patches 1802-1806 may be in the form of arc shapes that are positioned around the perimeter of the pressure applying surface 1216 to provide information regarding applied pressure and distribution of the pressure around the perimeter of the surface. As another example, pressure-sensitive film patches may be in the form of geometric shapes, such as triangles 1808 or circles 1810, that may be positioned in various locations on the pressure applying surface 1216.

In some embodiments, the pressure-sensitive film may be applied in the form of a ribbon or strip 1902 positioned along the periphery of the patient-facing surface of the pressure applying surface 1216 as illustrated in FIG. 19. Such a pressure-sensitive film ribbon or strip of 1902 may provide a visual indication of the pressure applied to the patient by the pressure applying surface without obstructing a view of the sutures through the transparent surface. In such embodiments, the pressure-sensitive film ribbon or strip 1902 may be positioned around the slit 1208 so that sutures can be passed through the vessel clamping pressure device 1700 as described above. In some embodiments, pressure-sensitive film ribbon or strip 1902 positioned adjacent to the outer perimeter portion of the pressure applying surface 1216 may have a radial width between about two millimeters and about five millimeters. In some embodiments, the pressure-sensitive film ribbon or strip 1902 positioned adjacent to the outer perimeter portion of the pressure applying surface 1216 may have a radial width of about three millimeters.

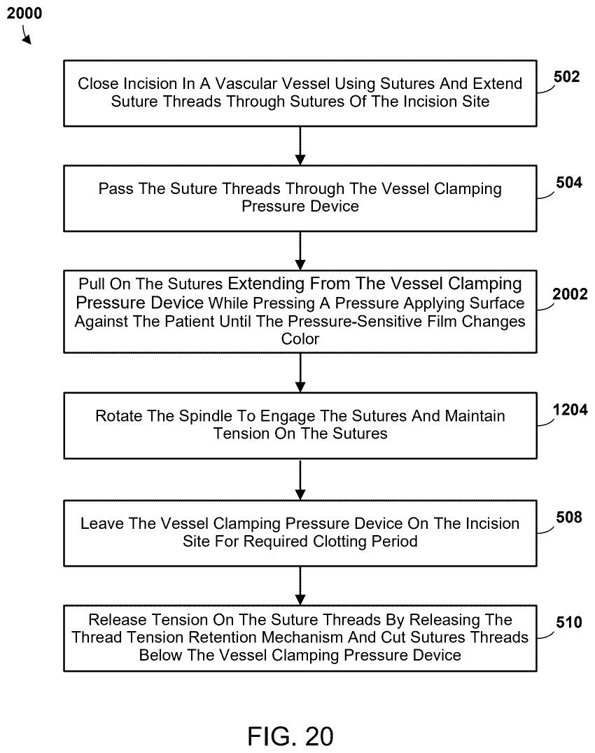

FIG. 20 illustrates a method 2000 of using the vessel clamping pressure device of the embodiment shown in FIGS. 17-19. In block 502, the clinician may close the incision in a vascular vessel using sutures and extend the sutures of the incision site as described above. In block 504, the clinician may pass the suture threads through slit 1206, 1208, 1214 in the vessel clamping pressure device as described herein. For example, by handling the sutures extending from the wound, the clinician may insert a broadside of an extent of those sutures through the slits 1206, 1208, 1214.

In block 2002, the clinician may pull on the sutures extending from the wound out from the vessel clamping pressure device while pressing a pressure applying surface (e.g., 106, 402, 1216) against the patient until the pressure-sensitive film 1702, 1802, 1804, 1806, 1808, 1810, 1902 changes color. In doing so, the clinician may increase the pressure applied to the patient until the color change reaches a color intensity indicative of sufficient applied pressure, such as by comparing the generated color to a color key. The clinician may also observe color changes on different portions of the pressure applying surface to confirm that pressure is being applied consistently across the surface.

Once the color change of the pressure-sensitive film indicates sufficient pressure is being applied to the patient, the clinician may rotate the spindle to engage the sutures and maintain tension on the sutures in block 1204 as described.

In block 508, the vessel clamping pressure device may be left on the incision site for the required clotting period, and in block 510, a clinician may release tension on the suture threads by releasing the thread tension retention mechanism and cut suture threads below the vessel clamping pressure device as described herein.

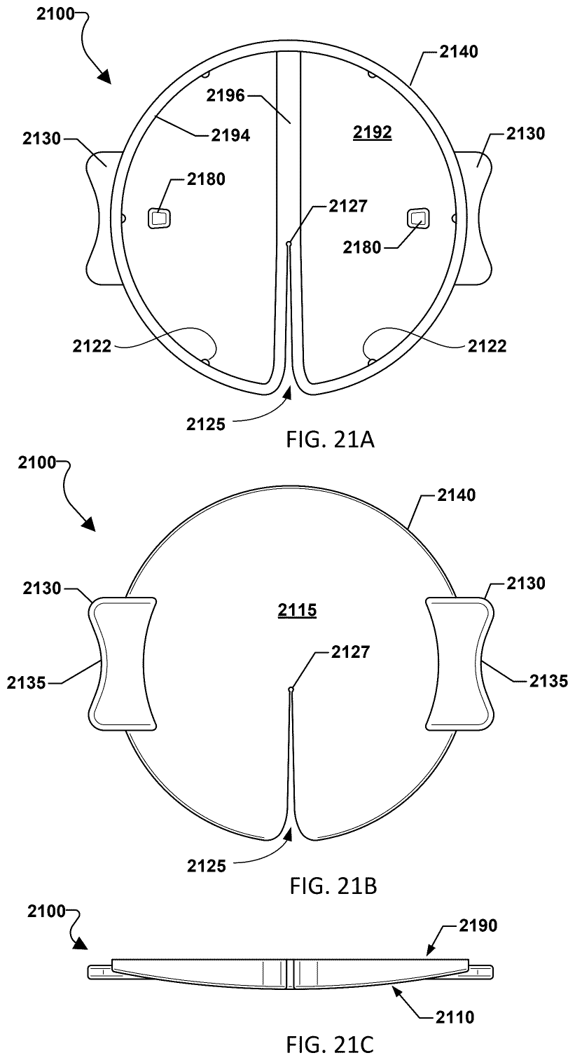

FIGS. 21A-21C illustrate various aspects of a base cover 2100 in accordance with further embodiments. FIG. 21A shows a top view of the base cover 2100, while FIG. 21B depicts a bottom view and FIG. 21C shows a side view of the base cover 2100. The base cover 2100 may be configured to interface with a vessel clamping pressure device (e.g., 1200) to provide a stable platform for applying pressure to a patient's skin while maintaining tension on suture threads.

As illustrated in FIGS. 21A-21C, the base cover 2100 may include a bottom 2110 having a bottom surface 2115 that may be configured to contact or at least face the skin of a patient. The bottom surface 2115 may be curved or contoured to more gently apply pressure to the skin of the patient, providing a comfortable interface between the vessel clamping pressure device and the patient's body. In some embodiments, the bottom surface 2115 may have a shape selected to match typical contours of a patient's body at locations where intravascular incisions are commonly made. Alternatively, the bottom 2110 may be generally flat, or portions thereof may be flat. In various embodiments, the curvature or lack thereof of the bottom 2110 may match the shape of the bottom of the pressure applying surface (e.g., 1216, 2216). Additionally, the bottom 2110 may be textured or otherwise configured to avoid slipping on the patient's skin (e.g., 302).

The base cover 2100 may include a base slit 2125 extending through the base cover 2100 from the bottom surface 2115 to an upper surface. The base slit 2125 may be configured to align with corresponding slits in other components of the vessel clamping pressure device to enable broadside insertion of suture threads. The base slit 2125 may include a base slit inner end 2127 that may define the extent of the slit within the base cover 2100. In some embodiments, the base slit 2125 may be sized to accommodate multiple suture threads simultaneously while providing sufficient engagement when the base cover 2100 is rotated relative to other components. The outermost portion of the base slit 2125 may include a flared portion, providing a wider inlet to the base slit 2125. In some embodiments, the overall base slit 2125 may have a tapered profile that widens toward the outer edge 2140. The base slit 2125 may extend from an outer edge 2140 of the base cover 2100 toward a central region, allowing suture threads to be inserted laterally into the vessel clamping pressure device. In various embodiments, the innermost portion of the base slit inner end 2127 lies radially outward of a radial center of the base cover 2100.

The bottom 2110 may be manufactured from biocompatible materials that are safe for contact with human skin and may be configured to distribute pressure evenly across the contact area. Such biocompatible materials may include medical-grade polymers, thermoplastics, silicones, or other materials that have been tested and approved for medical device applications involving direct skin contact. In some embodiments, the biocompatible materials may be selected from materials that comply with ISO 10993 standards for biological evaluation of medical devices, ensuring that the materials do not cause cytotoxicity, sensitization, irritation, or other adverse biological responses when in contact with patient tissue. The bottom 2110 may be manufactured from materials such as medical-grade polyethylene, polypropylene, polyurethane, silicone rubber, or thermoplastic elastomers that provide appropriate flexibility and durability for the intended application.

The configuration for distributing pressure evenly across the contact area may include surface texturing, material selection with appropriate durometer properties, or geometric design features that prevent pressure concentration at specific points. In some embodiments, the bottom 2110 may include a cushioning layer or padding material that enhances patient comfort while maintaining the structural integrity required for effective pressure application. The even pressure distribution may be achieved through the use of materials with controlled compression characteristics that deform uniformly under load, thereby spreading the applied force across the entire contact surface rather than creating localized pressure points that could cause patient discomfort or tissue damage.

The base cover 2100 may include grip wings 2130 extending from opposite sides of the base cover 2100, as shown in FIGS. 21A and 21B. Each grip wing 2130 may have a grip wing contour 2135 that may be configured to facilitate gripping by a user during positioning and manipulation of the vessel clamping pressure device. The grip wing contour 2135 may include textured surfaces, indentations, or raised areas that provide enhanced grip for a clinician's fingers. In some cases, the grip wings 2130 may extend laterally from the base cover 2100 to provide sufficient leverage for rotating the base cover 2100 relative to other components of the vessel clamping pressure device. The grip wings 2130 may be sized and positioned to allow a clinician to easily grasp the base cover 2100 with one hand while either manipulating the vessel clamping pressure device (e.g., 1200) or support structure (e.g., 1202, 2202) thereof or maintaining control of suture threads with the other hand.

The base cover 2100 may also include a top 2190 portion that may be configured to interface with other components of the vessel clamping pressure device. The top 2190 may include a base seat 2192 that may be configured to receive and hold a pressure applying element therein. The base seat 2192 may have a complementary shape to the pressure applying element, providing a secure connection while allowing for rotational movement between the base cover 2100 and the pressure applying element and the support structure attached thereto. A base rim 2194 may extend around the perimeter of the base seat 2192, providing structural support and defining the boundaries of the seating area.

The base rim 2194 may include an inner lip configured to trap the pressure applying element or an outer portion thereof, providing a snap-fit attachment feature that still allows the pressure applying element to rotate relative to the base cover 2100. The inner lip may extend radially inward from the base rim 2194 and may be configured to engage with a corresponding groove, flange, or outer edge of the pressure applying element (e.g., 2218). In some embodiments, the inner lip may be formed as a continuous circumferential feature that extends around the entire perimeter of the base rim 2194, providing uniform engagement with the pressure applying element (e.g., 2216). In some embodiments, the inner lip may be formed as discrete segments or tabs positioned at specific locations around the base rim 2194 to provide selective engagement points with the pressure applying element 2216.

The snap-fit attachment feature provided by the inner lip may allow for a secure mechanical connection between the base cover 2100 and the pressure applying element 2216 while permitting controlled rotational movement between these components. The inner lip may be configured with sufficient flexibility to allow the pressure applying element 2216 to be inserted into the base seat 2192 by applying a moderate force, causing the inner lip to deflect outward temporarily during insertion. Once the pressure applying element 2216 is fully seated, the inner lip may return to its original position, creating a mechanical interference that prevents inadvertent separation of the components during normal use.

In some embodiments, the inner lip may include a tapered or chamfered leading edge that facilitates insertion of the pressure applying element 2216 while providing a more pronounced retention feature once the components are fully engaged. The height and thickness of the inner lip may be selected to provide appropriate retention force while allowing for intentional disassembly when removal of the pressure applying element 2216 is desired. The material properties of the base cover 2100, particularly in the region of the inner lip, may be selected to provide appropriate flexibility and durability for repeated engagement and disengagement cycles if the device is configured for reuse rather than single-use disposal.

The base cover 2100 may further include detents 2180 positioned on the upper surface of the base cover 2100, as shown in FIG. 21A. The detents 2180 may be configured to resist rotation of the base cover 2100 relative to the pressure applying element until a threshold rotation force is applied, upon which the detents may permit an increment of rotation with a click. In some cases, the detents 2180 may be positioned at specific angular intervals to correspond to desired rotational orientations of the base cover 2100. The detents 2180 may engage with corresponding features on the pressure applying surface (i.e., the underside of the pressure applying element) to provide controlled resistance during rotation and to help maintain the base cover 2100 in selected rotational orientations when no rotational force is applied. A central rib 2196 may extend across the base seat 2192, providing additional structural support and potentially serving as a reference point for alignment with other components. The central rib 2196 may be positioned to interact with corresponding features on the pressure applying element, further enhancing the mechanical connection between components.

The detents 2180 may be configured to generate an audible click when they engage with corresponding recesses or features on the underside of the pressure applying element 2216. As the base cover 2100 is rotated relative to the pressure applying element, the detents 2180 may deflect slightly as they pass over raised surfaces or ridges on the pressure applying element, and then snap into corresponding recesses or depressions. This deflection and subsequent engagement may create a distinct clicking sound that provides audible feedback to the clinician, indicating that the components have reached the next rotational orientation. The detent locations and corresponding audible click may be positioned and configured to serve as a signal that the base cover 2100 has been rotated to the desired position for optimal suture thread engagement.

In addition to the audible feedback, the detents 2180 may provide tactile feedback through a locking-into-place sensation that the clinician can perceive through their fingers when gripping the grip wings 2130. When the detents 2180 engage with their corresponding features, the clinician may feel a slight resistance followed by a settling sensation as the detents snap into position. This tactile feedback may be accompanied by a momentary increase in rotational resistance followed by a decrease, creating a distinct feel that indicates proper positioning. The combination of audible and tactile feedback may help ensure that the clinician rotates the upper assembly to the correct position without over-rotation or under-rotation.

The detents 2180 may be designed with specific geometric features to enhance the feedback mechanisms. In some embodiments, the detents may have a ramped or angled leading surface that gradually increases resistance as rotation progresses, followed by a steeper trailing surface that provides the distinct snap-in sensation. The height, width, and spacing of the detents 2180 may be optimized to provide clear feedback while maintaining smooth operation. The material properties of the detents 2180 and the corresponding engagement features may be selected to provide appropriate flexibility for deflection while maintaining durability for repeated use.

Alternative mechanisms may be incorporated to provide similar positioning feedback functionality. In some embodiments, a spring-loaded ball detent system may be integrated into the base cover 2100, where one or more spring-biased balls are positioned to engage with corresponding dimples or recesses in the pressure applying element. The spring-loaded balls may provide consistent engagement force and clear tactile feedback as they move into and out of the engagement positions during rotation.

In some cases, a ratchet mechanism may be incorporated between the base cover 2100 and the pressure applying element, providing incremental positioning with distinct stops at predetermined angular intervals. The ratchet mechanism may include a series of teeth or ridges on one component that engage with a spring-loaded pawl or similar feature on the mating component. This arrangement may provide both audible clicks and tactile resistance at each incremental position, allowing the clinician to feel and hear each step of the rotation process.

Another alternative mechanism may include magnetic positioning elements, where small magnets embedded in the base cover 2100 align with corresponding magnetic elements or ferromagnetic materials in the pressure applying element. The magnetic attraction may provide a subtle but perceptible pull as the components approach the desired rotational orientation, followed by a settling sensation when proper alignment is achieved. The magnetic forces may be calibrated to provide sufficient feedback without interfering with the overall operation of the device.

In some embodiments, a friction-based positioning system may be employed, where raised ridges, bumps, or textured surfaces on the base cover 2100 engage with corresponding features on the pressure applying element. The friction-based system may provide continuous resistance during rotation with distinct changes in resistance at predetermined positions, allowing the clinician to feel when the optimal position has been reached through variations in the rotational force required.

The base cover 2100 may be configured to rotate relative to the pressure applying element from an open rotational orientation, in which the base slit 2125 is aligned with the slit in the pressure applying element, to a closed rotational orientation, in which the base slit 2125 is not aligned with the pressure applying element slit. This rotational capability allows a clinician to initially insert suture threads through aligned slits and then rotate the base cover 2100 to partially close the passage, thereby providing additional engagement and aligning the suture threads with the centerline of the device. The base cover 2100 may also include friction tabs 2122 that may provide additional gripping surfaces or may serve to increase friction between the base cover 2100 and the pressure applying element. These friction tabs 2122 may help prevent unwanted rotation between the vessel clamping pressure device once oriented relative to the base cover 2100 as desired.

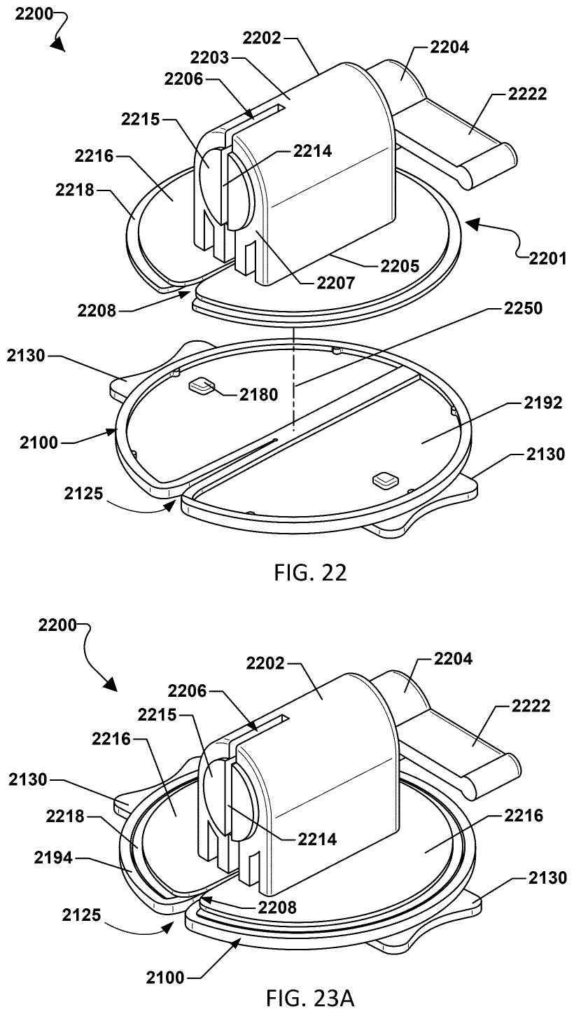

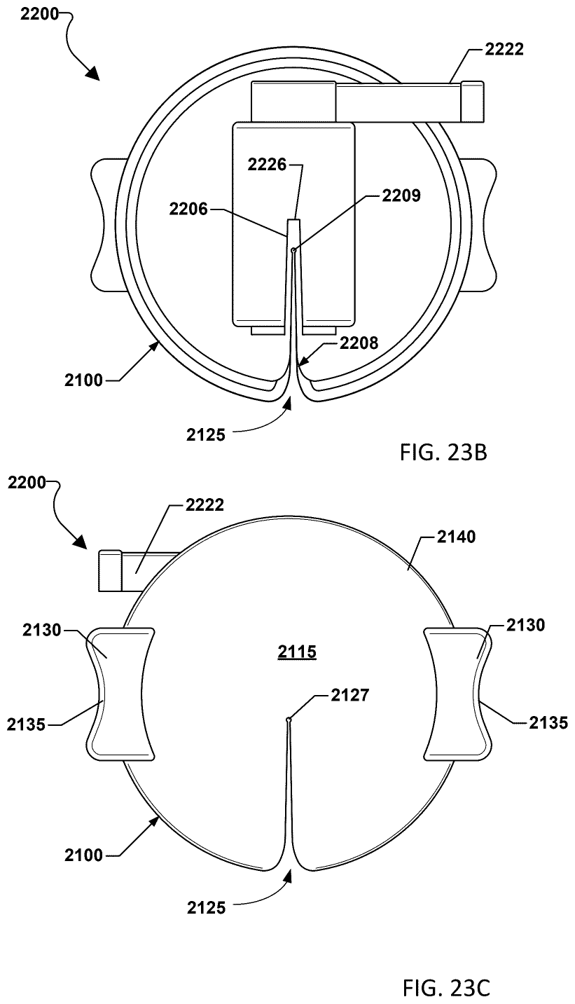

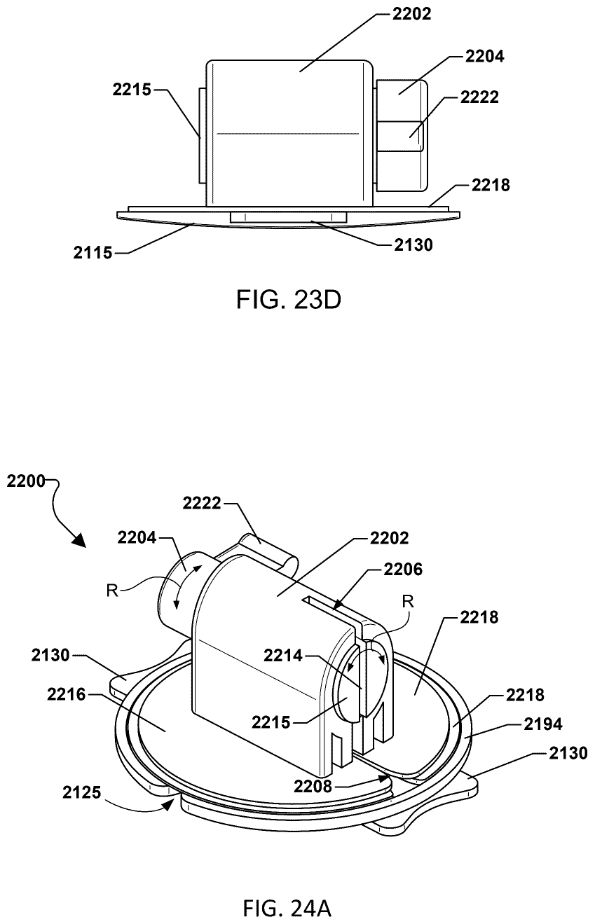

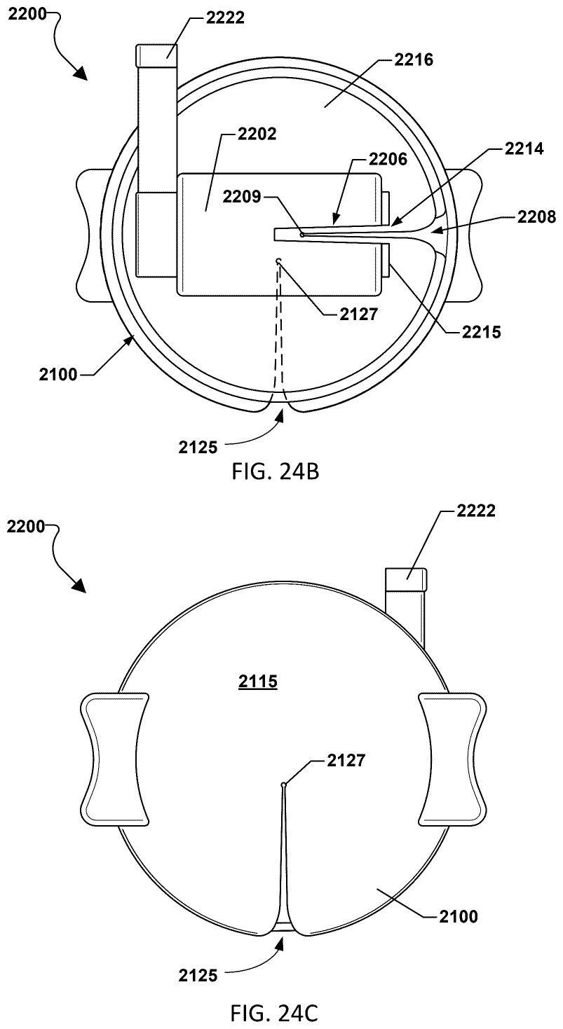

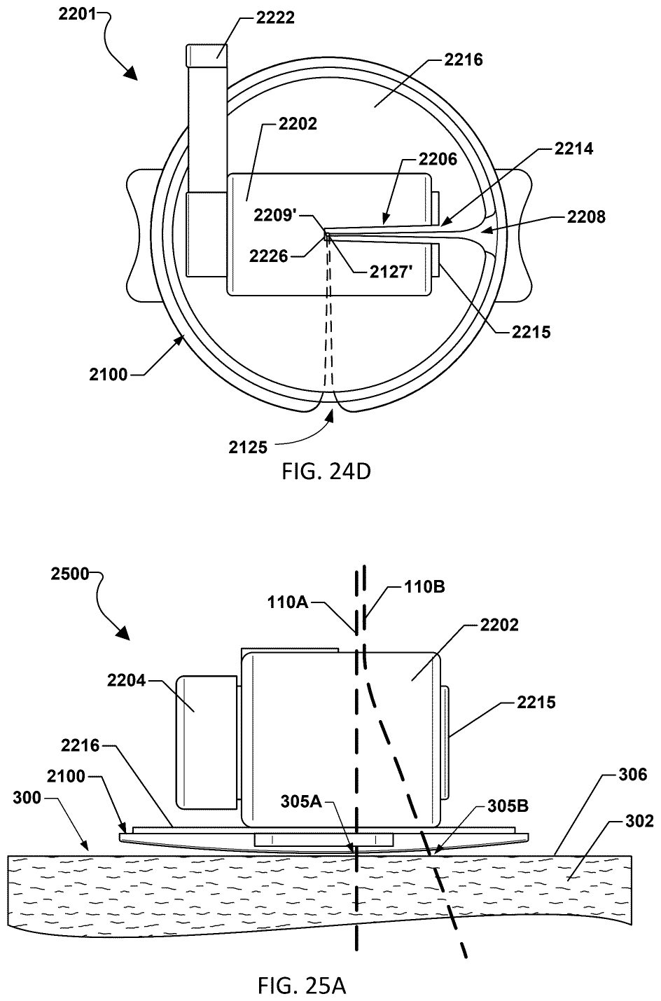

FIGS. 22-24C illustrate various aspects of the vessel clamping pressure assembly 2200 in accordance with various embodiments. FIG. 22 shows an exploded view of the vessel clamping pressure assembly 2200, while FIGS. 23A-23D show the assembled configuration of the vessel clamping pressure assembly 2200 in a fully open configuration. FIGS. 24A-24C show the assembled configuration of the vessel clamping pressure assembly 2200 in a partially open configuration. Differences between the fully open configuration and the partially open configuration are described below.

The vessel clamping pressure assembly 2200 combines a vessel clamping pressure device 2201, similar to those described above for prior embodiments, with the base cover 2100 to provide a vessel clamping pressure assembly 2200 with enhanced features that can maintain tension on suture threads while applying controlled pressure to a patient's suture incision. The assembled configuration may provide a complete functional unit that can be manipulated by a clinician to engage suture threads and maintain tension on closure sutures.

As illustrated in FIGS. 22-24C, the vessel clamping pressure assembly 2200 may include the vessel clamping pressure device 2201, which includes a support structure 2202 that may serve as a primary structural framework. The support structure 2202 may include a support structure slit 2206 extending completely through the support structure 2202 from a first side 2203 to a second side 2205 thereof. The support structure slit 2206 may interrupt a third side 2207 of the support structure 2202, in which the third side 2207 extends from the first side 2203 to the second side 2205. In this way, the support structure slit 2206 extends into the support structure 2202 from the third side 2207 to a support slit inner end 2226. This configuration allows the support structure slit 2206 to provide an open pathway for broadside insertion of suture threads into the vessel clamping pressure assembly 2200. The support structure 2202 may be manufactured from biocompatible materials and may be configured to provide structural integrity while maintaining the ability to interface with other components of the vessel clamping pressure device.

The vessel clamping pressure device 2201 may further include a spindle 2204 that is positioned within a passageway of the support structure 2202 and configured to rotate within the passageway. The spindle 2204 may include a spindle slit 2214 in a front face 2215 of the spindle 2204. The spindle slit 2214 may be configured to align with the support structure slit 2206 when the spindle 2204 is positioned in an open configuration. When the spindle slit 2214 and the support structure slit 2206 are aligned, suture threads may be inserted through both slits on a side of the device simultaneously (i.e., via a broadside insertion). The spindle 2204 may be configured to rotate within the support structure 2202 to move the spindle slit 2214 out of alignment with the support structure slit 2206, thereby engaging and binding suture threads that have been inserted through the aligned slits. The spindle 2204 may include a handle 2222, disposed on an opposite side from the front face 2215 of the spindle 2204. The handle 2222 may be used by a clinician to rotate the spindle 2204 around an axis perpendicular to a central axis 2250 of the support structure 2202.

The vessel clamping pressure device 2201 may include a pressure applying element, referred herein as a pressure applying surface 2216, fixedly secured to the second side 2205 of the support structure 2202. Alternatively, the pressure applying surface 2216 may be integrally formed with the support structure 2202. The pressure applying surface 2216 may include a surface slit 2208 that extends therethrough and interrupts an outer perimeter of the pressure applying surface 2216, extending radially inward to a surface slit inner end 2209. As shown in FIG. 23B, the surface slit inner end 2209 may not extend as far inward as the support slit inner end 2226. Since the surface slit 2208 is aligned with the support structure slit 2206, the two slits 2206, 2208 provide a continuous passageway for receiving suture threads, particularly when the spindle slit 2214 is in rotational alignment with the two slits 2206, 2208.

The pressure applying surface 2216 may include a bottom side configured to contact or face the base cover 2100. The pressure applying surface 2216 may include an outer lip 2218 that extends around the perimeter of the pressure applying surface 2216, providing structural definition and potentially serving as a seating interface for engagement with the base cover 2100. Alternatively, the pressure applying surface 2216 may have a more constant thickness, without a thinner outer lip 2218.

The integration of these components within the vessel clamping pressure assembly 2200 provides a device for maintaining tension on suture threads to apply pressure to a sutured incision, relieving the need for a caregiver to continue to apply pressure after the incision site is sutured. The vessel clamping pressure device 2201 may be configured to engage with the base cover 2100 through a snap-fit connection that secures the components while maintaining rotational capability. The pressure applying surface 2216 may be dimensioned to fit within the base seat 2192 of the base cover 2100, with the outer lip 2218 of the pressure applying surface 2216 configured to engage with the inner lip of the base rim 2194. During assembly, the pressure applying surface 2216 may be positioned above the base seat 2192 and pressed downward with moderate force, causing the inner lip of the base rim 2194 to deflect outward temporarily as the outer lip 2218 passes through the opening. Said another way, by lowering the vessel clamping pressure device 2201 along the axis 2250, the vessel clamping pressure device 2201 can be snapped into the base seat 2192.

Once the pressure applying surface 2216 is fully inserted into the base seat 2192, the inner lip of the base rim 2194 may return to its original position, creating a mechanical interference that captures the outer lip 2218 and prevents vertical separation of the components. The snap-fit engagement may create an audible or tactile indication when the pressure applying surface 2216 is properly seated within the base cover 2100. The complementary shapes of the base seat 2192 and the pressure applying surface 2216 may allow the vessel clamping pressure device 2201 to rotate freely within the base cover 2100 while the inner lip maintains the mechanical connection.

The rotational movement between the vessel clamping pressure device 2201 and the base cover 2100 may be facilitated by the smooth interface between the outer lip 2218 and the inner lip of the base rim 2194. The detents 2180 positioned on the upper surface of the base cover 2100 may engage with corresponding features on the bottom side of the pressure applying surface 2216 to provide controlled resistance during rotation and to help maintain the vessel clamping pressure device 2201 in selected rotational orientations relative to the base cover 2100. This snap-fit connection may allow the vessel clamping pressure device 2201 to be securely attached to the base cover 2100 during use while permitting the rotational adjustment needed to engage suture threads through the misalignment of the surface slit 2208 and the base slit 2125.

The alignment of the support structure slit 2206, the spindle slit 2214, and the surface slit 2208 may enable broadside insertion of suture threads, eliminating the need to axially thread sutures through narrow openings or holes. The rotational capability of the spindle 2204 within the support structure 2202 may allow a clinician to initially position the vessel clamping pressure assembly 2200 with all slits aligned for easy suture insertion, and then rotate the spindle 2204 to bring the sutures together so that they can be wound on the spindle to maintain even tension on the individual suture threads. The handle 2222 may provide leverage for this rotational movement within the support structure 2202.

FIGS. 23A-23D illustrate various aspects of the vessel clamping pressure assembly 2200 in an assembled and fully open configuration. In contrast, FIGS. 24A-24C show the vessel clamping pressure assembly 2200 in a rotated configuration with the spindle 2204 open. This rotation plays a role in the device's functionality, particularly in how it interacts with suture threads through the alignment and misalignment of slits 2206, 2208, 2214, 2125. The rotated configuration refers to the vessel clamping pressure device 2201 being rotated away from the fully open orientation (i.e., greater than zero degrees and less than 360 degrees) relative to the base cover 2100 so that the support structure slit 2206 and the surface slit 2208 are no longer aligned with the base cover slit 2125. The term “open spindle” or reference to the spindle 2202 being “open” refers to the spindle slit 2214 being aligned with the support structure slit 2206 and the surface slit 2208. The term “closed spindle” or reference to the spindle 2202 being “closed” refers to any orientation in which the spindle slit 2214 is not aligned with the support structure slit 2206 and the surface slit 2208. These figures demonstrate the spatial relationships between components and how the vessel clamping pressure device appears when components are positioned in different orientations, which correspond to different stages of operational use.

The base slit 2125 in the base cover 2100 forms a cut-out extending radially inward from the outer edge 2140 toward a central region. Similarly, the surface slit 2208 of the pressure applying surface 2216 and the support structure slit 2206 of the support structure 2202 together form another cut-out extending radially inward from their outer perimeters. Additionally, the spindle slit 2214 forms another cut-out that can be rotated to align with the support structure slit 2206 and the surface slit 2208. Initially, when the support structure slit 2206, the surface slit 2208, and the spindle slit 2214 are aligned with the base slit 2125 (e.g., FIGS. 23A-23D), the four slits together create a continuous open passageway that allows for the broadside insertion of suture threads directly into the aligned slits without needing to thread them end-first.

As the support structure 2202 is rotated relative to the base cover 2100, the support structure slit 2206, the surface slit 2208, and the spindle slit 2214 together pivot progressively out of alignment with the base slit 2125, gradually closing off the radially outer portion of the passageway and thereby trapping any inserted suture threads within the remaining inner opening to prevent their lateral escape. Upon sufficient rotation, the support structure slit 2206, the surface slit 2208, and the spindle slit 2214 become fully misaligned with the base slit 2125 (e.g., FIGS. 24A-24C), causing the suture threads extending through them to be drawn together and clamped between the interfacing surfaces of the pressure applying surface 2216 and the base cover 2100, effectively holding them in place. The degree of clamping or holding force applied to the suture threads depends upon the manufacturing tolerances and dimensional relationships between the upper surface of the base cover 2100 and the lower surface of the pressure applying element 2216. Tighter tolerances between these interfacing surfaces create a narrower gap that applies greater compressive force to the suture threads, resulting in stronger clamping action and more secure retention of the threads. Conversely, looser tolerances provide a wider gap that applies less compressive (i.e., holding) force, allowing the clinician to adjust the tension of the suture threads extending from the wound site. In some embodiments, a significant gap is maintained between the upper surface of the base cover 2100 and the lower surface of the pressure applying element 2216 in order to avoid any clamping or other force that holds the suture threads in place in order to give the clinician more freedom to adjust tension before turning the spindle 2204 to provide a clamping force on the suture threads (e.g., 110).

FIG. 24B, which illustrates the vessel clamping pressure device 2201 rotated ninety degrees from the fully open orientation (e.g., FIG. 23B), shows how a position of the surface slit inner end 2209 can be offset from a position of the base cover slit inner end 2127. Rotation from the fully open position pivots the surface slit 2208 and its surface slit inner end 2209 out of alignment with the base cover slit 2125 and its base cover slit inner end 2127.

FIG. 24D, which illustrates an alternative vessel clamping pressure device 2201 in a similar rotational configuration, shows how the extended surface slit inner end 2209′ and the extended base cover slit inner end 2127′ may maintain alignment even when the device is rotated from the fully open orientation. In contrast to the configuration shown in FIG. 24B, where the surface slit inner end 2209 becomes offset from the base cover slit inner end 2127 upon rotation, the extended slits in FIG. 24D may extend to or beyond the radial center of the device. This extended configuration may allow the extended surface slit inner end 2209′ and the extended base cover slit inner end 2127′ to remain aligned at the radial center of the device, creating a small aligned portion that maintains continuity between the surface slit 2208 and the base cover slit 2125. The extended length of these slits may eliminate the offset condition that occurs with shorter slits, ensuring that suture threads can continue to pass through the central aligned portion even when the outer portions of the slits are rotated out of alignment. This design may provide improved suture thread management by maintaining a central pathway while still allowing the rotational engagement mechanism to function effectively.

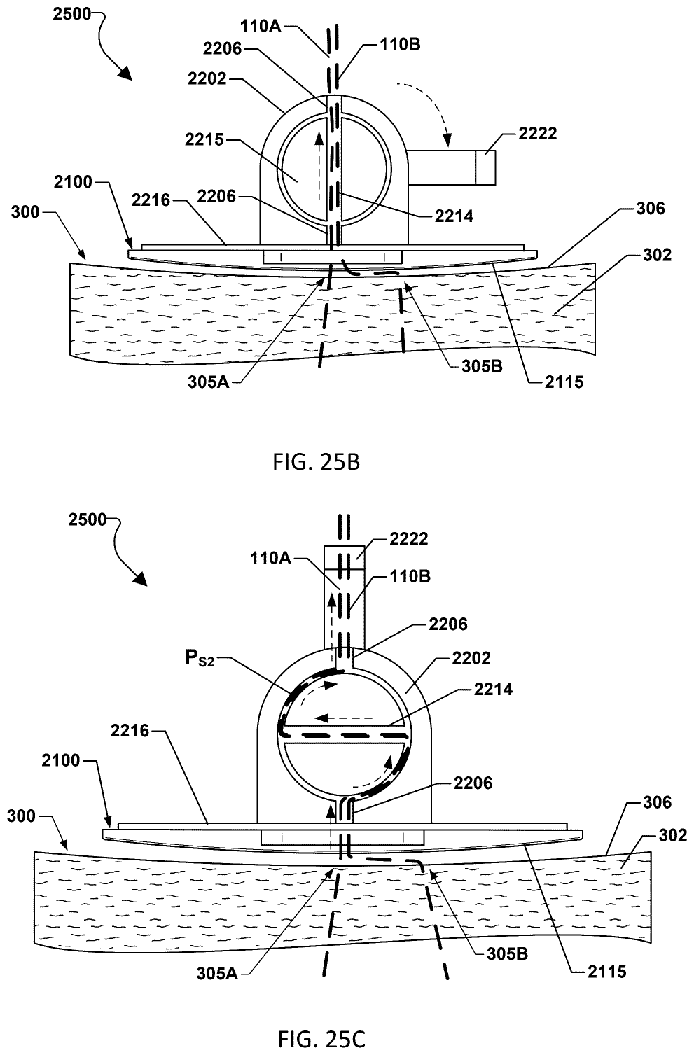

FIGS. 25A-25C provides a side elevation view of an applied assembly 2500 positioned on a patient 300 in an operational configuration. The applied assembly 2500 includes the vessel clamping pressure assembly 2200 positioned over a wound site on the patient 300. The base cover 2100 may be positioned with its bottom surface 2115 in direct contact with the skin 302 of the patient 300, providing a stable interface between the device and the patient's body surface. FIGS. 25A-25C show a small gap between the bottom surface 2115 and the skin 302 for illustrative purposes for better visualization of the path of the suture threads 110A, 110B.

FIG. 25A shows how the first suture 110A and the second suture 110B may extend from the skin surface 306 of the patient 300, emerging from exit sites 305A and 305B, respectively. These suture threads 110A, 110B may pass through the aligned slits of the device, including the base slit 2125, the surface slit 2208, the support structure slit 2206, and the spindle slit 2214, demonstrating the broadside insertion capability of the vessel clamping pressure assembly 2200. The suture threads 110A, 110B may extend upward through the device, allowing for tension adjustment and secure engagement when initially the vessel clamping pressure device 2201 is rotated relative to the base cover 2100 and subsequently the spindle 2204 is rotated relative to the support structure 2202 to the closed configuration. The skin surface 306 may be visible beneath the pressure applying surface 2216 and the base cover 2100, showing the interface between the device and the patient's body at the sutured incision. The configuration shown in FIG. 25A represents the initial positioning of the device when the broadside insertion of the suture threads 110A, 110B may be performed.

FIG. 25B illustrates the assembly 2500 in an intermediate configuration following the initial positioning shown in FIG. 25A. In this configuration, the vessel clamping pressure device 2201 has been rotated relative to the base cover 2100, causing the support structure slit 2206, the surface slit 2208, and the spindle slit 2214 to move out of alignment with the base slit 2125. This rotational movement may create a progressive narrowing of the passageway through which the first suture 110A and the second suture 110B extend, gently guiding the suture threads toward each other without applying significant tension to the underlying wound sites at exit site A 305A and exit site B 305B.

The gradually increasing misalignment of the slits cause the suture threads 110A, 110B to be gradually drawn together as they pass through the increasingly restricted opening between the pressure applying surface 2216 and the base cover 2100. This gentle convergence of the suture threads may occur without pulling on the patient's skin 302 because the rotational movement primarily affects the alignment of the suture threads rather than applying direct tension to the suture threads extending from the sutured incision. The bottom surface 2115 of the base cover 2100 may remain in stable contact with the skin surface 306, maintaining consistent positioning while the internal components guide the suture threads into alignment.

This intermediate configuration may prepare the suture threads 110A, 110B for more effective engagement in the subsequent stage by positioning them closer together within the narrowed passageway. The gradual nature of this transition may allow the clinician to control the degree of thread convergence while maintaining the ability to make fine adjustments before proceeding to the final clamping stage. The support structure 2202 itself may provide the clinician with a type of handle to precisely control the rotational orientation, enabling smooth progression from the fully open configuration of FIG. 25A to this intermediate state that optimizes the suture thread arrangement for secure retention in the following operational phase.

FIG. 25C illustrates the applied assembly 2500 in a final operational configuration following the intermediate stage shown in FIG. 25B. In this third stage, the spindle 2204 has been rotated relative to the support structure 2202 around an axis that is perpendicular to the rotational axis used when the support structure 2202 was rotated relative to the base cover 2100 in the previous stage. This spindle rotation moves the spindle slit 2214 out of alignment with the support structure slit 2206 and the surface slit 2208, creating a binding engagement that securely clamps the first suture 110A and the second suture 110B within the vessel clamping pressure assembly 2200.

The rotation of the spindle 2204 may cause the suture threads 110A, 110B to be pressed between the spindle 2204 and the interior surfaces of the support structure 2202. As shown in FIG. 25C, the suture threads 110A, 110B are bent through a serpentine path that prevents them from sliding through the device while maintaining the desired tension between the applied assembly 2500 and the sutured incision at exit site 305A and exit site 305B. This clamping action may effectively lock the suture threads in position, ensuring that the pressure applying surface 2216 continues to press against the skin surface 306 of the patient 300 with consistent force throughout the healing period.

The spindle rotation may be performed to maintain the therapeutic pressure applied to the wound site without requiring continuous manual intervention by the clinician. By securing the suture threads 110A, 110B in this manner, the applied assembly 2500 may provide sustained compression that promotes hemostasis and supports the natural clotting process at the wound sites. The handle 2222 may remain accessible to the clinician during this stage, allowing for potential adjustments to the spindle position if needed while the device remains in place on the patient 300. In this final clamping configuration, the patient may be moved from the surgical area to recovery while the vessel clamping pressure assembly 2200 continues to provide the necessary pressure to prevent bleeding through the sutured incision and enable wound closure.

After the applied assembly 2500 has maintained the configuration shown in FIG. 25C for at least a desired clotting period, the removal process may be initiated by reversing the engagement sequence. The spindle 2204 may be rotated back from its closed configuration to realign the spindle slit 2214 with the support structure slit 2206 and the surface slit 2208, returning the assembly to the intermediate configuration shown in FIG. 25B. This rotation may release the binding engagement between the spindle 2204 and the suture threads 110A, 110B, allowing the threads to move more freely within the narrowed passageway while still being guided by the partially misaligned slits.

Following the spindle rotation to release the binding engagement, the support structure 2202 may be rotated relative to the base cover 2100 to return all slits to full alignment, recreating the fully open configuration initially shown in FIG. 25A. This rotational movement may progressively widen the passageway as the support structure slit 2206, the surface slit 2208, and the spindle slit 2214 move back into alignment with the base slit 2125. The gradual realignment may allow the suture threads 110A, 110B to separate and return to their original positions without applying sudden tension to the wound sites at exit site 305A and exit site 305B.

Once all four slits are fully aligned, the suture threads 110A, 110B may be completely released from the vessel clamping pressure assembly 2200 through broadside removal. The clinician may lift the applied assembly 2500 away from the patient's skin 302, allowing the suture threads to slide laterally out of the aligned slits without requiring the threads to be pulled through narrow openings. This broadside removal capability may provide the same convenience during device removal as it does during initial application, eliminating the need for complex threading procedures.

After the vessel clamping pressure assembly 2200 has been removed, the clinician may address the protruding suture threads 110A, 1101B that remain extending from the wound sites. The clinician may cut the suture threads close to the skin surface 306 using surgical scissors or other appropriate cutting instruments. In some cases, the clinician may tie off the suture threads before cutting them, depending on the specific closure technique used and the healing progress observed at the wound sites. Alternatively, if the sutures were placed as temporary closure aids, the clinician may remove them entirely by gently pulling them from the sutured incision after confirming that adequate healing has occurred during the clotting period.

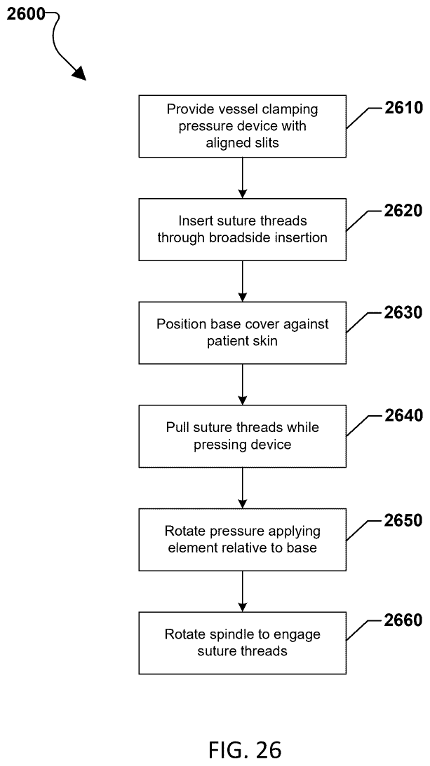

FIG. 26 is a process flow diagram illustrating a method 2600 for using a vessel clamping pressure device. The method 2600 may begin in block 2610, where a vessel clamping pressure assembly 2200 with aligned slits may be provided, for example by a clinician and/or assistant. In block 2610, the vessel clamping pressure device may include the support structure 2202 with the support structure slit 2206, the spindle 2204 positioned within a passageway of the support structure 2202 and including the spindle slit 2214, the pressure applying surface 2216 coupled to the support structure 2202 and including the surface slit 2208, and the base cover 2100 having the base slit 2125. The alignment of the support structure slit 2206, the spindle slit 2214, the surface slit 2208, and the base slit 2125 into the fully open configuration may enable broadside insertion of a stretched portion of suture threads therein, eliminating the need for threading sutures through narrow openings or holes.

In block 2620, the suture threads may be inserted into the vessel clamping pressure device through broadside insertion. In block 2620, the first suture (e.g., 110A) and the second suture (e.g., 110B) closing the wound may be inserted through broadside insertion into the aligned slits of the vessel clamping pressure device. The broadside insertion capability may allow the first and second sutures to be positioned laterally into the aligned slits when the sutures are stretched between the exit site 305A and exit site 305B on the skin surface 306 of the patient 300. The base slit 2125 extending through the base cover 2100 may provide the initial entry point for the first and second sutures, allowing the sutures to pass from the skin surface (e.g., 306) through the bottom surface (e.g., 2115) of the base cover (e.g., 2100). The alignment of all slits may create a continuous passageway that accommodates simultaneous insertion of multiple suture threads without requiring complex threading procedures.

In block 2630, the base cover may be positioned against the skin of the patient over a sutured incision. In block 2630, positioning the base cover against the skin may include gripping the grip wings (e.g., 2130) to facilitate positioning of the vessel clamping pressure device. The grip wings may extend from opposite sides of the base cover and may include the grip wing contours configured to facilitate gripping by a user during positioning and manipulation of the vessel clamping pressure device. The bottom surface (e.g., 2115) of the base cover may be curved to more gently apply pressure to the skin 302 of the patient 300, providing a comfortable interface between the vessel clamping pressure device and the patient's body. The curved or contoured shape of the bottom surface 2115 may conform to the natural contours of the patient's body at the wound site, reducing pressure points and enhancing patient comfort during the healing period.

In block 2640, the clinician may pull on the suture threads while pressing the device. In block 2640, the first and second suture threads may be gently pulled to tension the suture threads while pressing the vessel clamping pressure device against the patient. The tensioning of the suture threads may better position them relative to the vessel clamping pressure device before the subsequent blocks. The base cover 2100 may remain in contact with the skin 302 throughout this tensioning process, providing a stable platform for later pressure application while the internal components prepare to engage the sutures. The grip wings 2130 may provide leverage for maintaining control of the vessel clamping pressure device while applying tension to the suture threads with the other hand.

In block 2650, the vessel clamping pressure device (e.g., 2201) may be rotated relative to the base cover (e.g., 2100). Alternatively, the base cover may be rotated relative to the vessel clamping pressure device. In block 2650, the rotation of the pressure applying surface (e.g., 2216) and the support structure (e.g., 2202) relative to the base cover may partially or fully close the passage for the suture threads. This rotation may move the upper slits out of alignment with the base slit (e.g., 2125), creating a narrower passage for the suture threads to extend through the base cover and the vessel clamping pressure device. The base cover 2100 may include detents 2180 positioned on the upper surface of the base cover 2100, and rotating the pressure applying surface 2216 and the support structure 2202 relative to the base cover 2100 may include using the detents 2180 to determine where to stop rotating the pressure applying surface 2216 relative to the base cover 2100. The detents 2180 may provide tactile feedback to a clinician during operation, when the detents 2180 slide into matching recesses in the lower surface of the pressure applying surface 2216 to provide controlled resistance during rotation.

In block 2660, the clinician may rotate the spindle (e.g., 2204) to engage suture threads. In block 2660, the spindle may be rotated relative to the support structure to engage the suture threads and maintain tension thereon. The rotation of the spindle from an open configuration to a closed configuration may move the spindle slit (e.g., 2214) out of alignment with the support structure slit 2206, creating a binding engagement with the suture threads that have been inserted through the aligned slits. The handle (e.g., 2222) may provide leverage for rotating the spindle within the support structure, allowing a clinician to apply rotational force while maintaining control of the vessel clamping pressure assembly (e.g., 2200). The binding action created by the misalignment of the spindle slit with the support structure slit may prevent the suture threads from sliding through the vessel clamping pressure device while maintaining tension between the device and the wound sites at the exit sites (e.g., 305A, 305B).

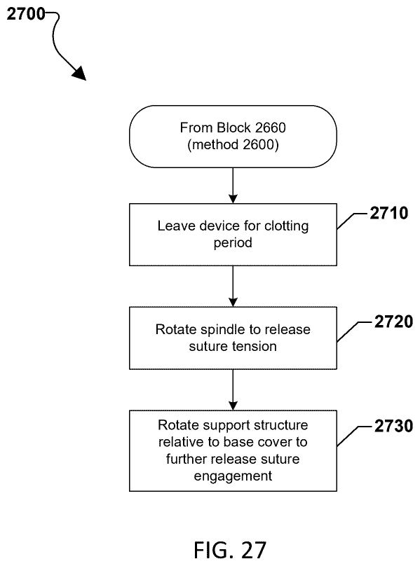

FIG. 27 is a process flow diagram illustrating a method 2700 for using a vessel clamping pressure device. The method 2700 may follow the operations of the method 2600, described above, after the healing period has been completed and the wound site has achieved adequate clotting. Following block 2660, the device may be left for a clotting period in block 2710. The vessel clamping pressure device may remain in position on the patient for a predetermined clotting period that allows the wound site to achieve hemostasis and begin the healing process. During the clotting period, the suture threads may maintain tension between the vessel clamping pressure device and the sutured incision at the exit sites 305A, 305B, ensuring consistent pressure application through the pressure applying surface against the skin of the patient.

The clotting period in block 2710 may vary depending on the specific medical procedure, the patient's condition, and the clinician's assessment of the wound healing progress. In some cases, the clotting period may range from several minutes to several hours, allowing sufficient time for the formation of stable blood clots at the wound sites and the initiation of the natural healing process. During the clotting period, the base cover may remain in contact with the skin of the patient through the bottom surface, providing a stable platform for pressure application to the sutured incision while the internal components maintain engagement with the suture threads. The spindle may remain in the closed configuration with the spindle slit misaligned with the support structure slit, ensuring that the binding engagement with the suture threads continues throughout the clotting period. The handle may remain accessible during the clotting period, allowing for potential adjustments if needed while maintaining the rotational orientation of the spindle within the support structure.

Following the completion of the clotting period, in block 2720, the spindle may be rotated from the closed configuration back to an open configuration to disengage the suture threads from the binding engagement within the vessel clamping pressure device, thereby releasing tension on the sutures. Releasing tension on the sutures releases the pressure applied to the wound sites through the pressure applying surface. The rotation of the spindle in block 2720 realigns the spindle slit with the support structure slit, creating a continuous passageway that allows the suture threads to move freely through the vessel clamping pressure device without binding or restriction. The handle may provide leverage for this rotational movement, allowing a clinician to apply controlled rotational force to return the spindle to the open configuration.

In block 2730, a support structure may be rotated relative to a base cover to further release suture engagement. In block 2730, the support structure may be rotated relative to the base cover to realign the surface slit with the base slit, creating a fully open passageway for the suture threads to be withdrawn from the vessel clamping pressure device. The rotation of the support structure relative to the base cover may reverse the partial closure of the passage that was created in block 2650 of the method 2600, returning the base slit to full alignment with the surface slit. The grip wings extending from opposite sides of the base cover may provide leverage for maintaining control of the base cover while the support structure is rotated to the open rotational orientation. The detents positioned on the upper surface of the base cover may provide tactile feedback during the rotation process, helping the clinician identify when the support structure has been returned to the proper alignment with the base cover.

The systematic disengagement process in the method 2700 may reduce the tension on the suture threads, ensuring that the vessel clamping pressure device can be safely removed from the patient without causing trauma to the sutured incision or disrupting the healing process that has occurred during the clotting period. These components may be sufficiently transparent to allow a clinician to observe the position and condition of the suture threads throughout the removal process, ensuring that the sutures are properly disengaged before the vessel clamping pressure device is completely withdrawn from the sutured incision. The visualization capability may provide additional safety and precision during the removal procedure, allowing the clinician to confirm that the sutures remain intact and properly positioned as the vessel clamping pressure device is disengaged.

Once the support structure has been rotated relative to the base cover to fully align all slits in block 2730, the suture threads may be free to move through the vessel clamping pressure device without restriction, allowing the device to be lifted away from the skin surface of the patient. The bottom surface of the base cover may be gently separated from the skin of the patient, completing the removal process while maintaining the integrity of the wound sites at the exit sites.

The methods and vessel clamping pressure devices of various embodiments may be used in a wide range of surgical procedures, particularly procedures that involve penetration or surgery on an artery or vein. Some non-limiting examples of surgical procedures for which various embodiments are well suited include minimally invasive catheter-based diagnostic and surgical procedures, including access sites for intracardial electrophysiology exam, cardiothoracic surgery, angioplasty and stenting, transcatheter aortic valve replacement (TAVR), catheter ablation, balloon valvuloplasty, vascular and endovascular surgery, endovascular aneurysm repair (EVAR/TEVAR), atherectomy, neurointerventional surgery, cerebral aneurysm coiling, mechanical thrombectomy for stroke, carotid artery stenting, and transurethral resection of the prostate (TURP). Additionally, the methods and vessel clamping pressure devices of various embodiments may be used to close incision sites for procedures involving the treatment of thrombosis, including thrombectomy, catheter-directed thrombolysis, vascular bypass, and vena cava filter placement, all of which require suturing of major blood vessels.

The foregoing method descriptions and the process flow diagrams are provided merely as illustrative examples and are not intended to require or imply that the operations of various embodiments must be assembled or performed in the order presented. As will be appreciated by one of skill in the art the order of operations in the foregoing embodiments may be performed in any order. Words such as “thereafter,” “then,” “next,” etc. are not intended to limit the order of the operations; these words are used to guide the reader through the description of the methods. Further, any reference to claim elements in the singular, for example, using the articles “a,” “an,” or “the” is not to be construed as limiting the element to the singular.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. A vessel clamping pressure device, comprising:
a pressure applying surface including a slit through which suture threads can be passed, the pressure applying surface being configured to apply pressure to skin of a patient at a site of a wound closure;
a support structure coupled to the pressure applying surface, the support structure comprising a through-hole and a slit through which suture threads can be passed; and
a spindle positioned within the through-hole of the support structure, wherein the spindle is configured to rotate within the through-hole and includes a slit through which suture threads can be passed.

2. The vessel clamping pressure device of claim 1, wherein the pressure applying surface includes a pressure-sensitive film configured to change color in response to the pressure applied to the skin of the patient by the pressure applying surface.

3. The vessel clamping pressure device of claim 2, wherein the pressure-sensitive film is applied in a strip adjacent to the perimeter of the pressure applying surface.

4. The vessel clamping pressure device of claim 2, wherein the pressure-sensitive film is configured one or more patches that are applied to the pressure applying surface.

5. The vessel clamping pressure device of claim 2, wherein the pressure-sensitive film is applied to a patient facing surface of the pressure applying surface so that the pressure-sensitive film contacts the skin of the patient when the vessel clamping pressure device is applied to the patient.

6. A vessel clamping pressure device, comprising:
a support structure including a support structure slit extending completely through the support structure from a first side to a second side thereof, wherein the support structure slit interrupts a third side of the support structure, wherein the third side extends from the first side to the second side;
a spindle positioned within a passageway of the support structure and configured to rotate within the passageway, the spindle including a spindle slit that is configured to align with the support structure slit, wherein rotation of the spindle rotates the spindle slit relative to the support structure from at least one open configuration to at least one closed configuration, wherein in the open configuration the spindle slit is aligned with the support structure slit and in the closed configuration the spindle slit is not aligned with the support structure slit;
a pressure applying element coupled to the second side of the support structure, the pressure applying surface including a pressure applying element slit aligned with the support structure slit; and
a base cover having a bottom surface configured to contact or at least face skin of a patient and an upper surface configured to be removably attached to the pressure applying element, the base cover including a base slit extending through the base cover, wherein the base cover is configured to rotate relative to the pressure applying element from an open rotational orientation in which the base slit is fully aligned with the pressure applying element slit to a closed rotational orientation in which the base slit is not fully aligned with the pressure applying element.

7. The vessel clamping pressure device of claim 6, wherein the base cover includes grip wings extending from opposite sides of the base cover.

8. The vessel clamping pressure device of claim 7, wherein each grip wing has a grip wing contour configured to facilitate gripping by a user.

9. The vessel clamping pressure device of claim 6, wherein the base cover includes a seat configured to receive and hold the pressure applying element therein.

10. The vessel clamping pressure device of claim 6, wherein the base cover includes detents positioned on the upper surface of the base cover, wherein the detents are configured to resist the rotation of the base cover relative to the pressure applying element.

11. The vessel clamping pressure device of claim 6, wherein the pressure applying element is sufficiently transparent to enable visualization of suture threads and skin beneath the pressure applying surface.

12. The vessel clamping pressure device of claim 6, wherein the base cover is sufficiently transparent to enable visualization of suture threads and skin beneath the base cover.

13. The vessel clamping pressure device of claim 6, wherein the spindle is configured to rotate to engage suture threads extending through the support structure slit, the spindle slit, the pressure applying element slit, and the base cover slit.

14. The vessel clamping pressure device of claim 6, wherein the rotation of the base cover relative to the pressure applying element rotates the pressure applying element slit relative to the base cover slit, leaving a narrower passage for the suture threads to extend through the base cover and the pressure applying element.

15. The vessel clamping pressure device of claim 7, wherein the rotation of the spindle from the open configuration to the closed rotational configuration is configured to bind suture threads between the spindle and the support structure with the passageway.

16. A method of applying pressure to a sutured incision, comprising:
providing a vessel clamping pressure device having a support structure including a support structure slit, a spindle positioned within a passageway of the support structure and including a spindle slit, a pressure applying element coupled to the support structure and including a pressure applying element slit, and a base cover having a base slit, wherein alignment of the support structure slit, the spindle slit, the pressure applying element slit, and the base slit enables broadside insertion of a stretched portion of suture threads therein;
inserting suture threads extending from the sutured incision through a broadside insertion into the aligned slits of the vessel clamping pressure device;
positioning the base cover against the skin of a patient over the sutured incision;
pulling the suture threads to tension the suture threads while pressing the vessel clamping pressure device against the patient;
rotating the pressure applying element and support structure relative to the base cover to partially close the passage for the suture threads; and
rotating the spindle relative to the support structure to engage the suture threads and maintain tension on the suture threads.

17. The method of claim 16, wherein the base cover includes grip wings extending from opposite sides of the base cover, and wherein positioning the base cover against the skin includes gripping the grip wings to facilitate positioning of the vessel clamping pressure device, and wherein a lower surface of the base cover is curved to more gently apply pressure to the skin of the patient.

18. The method of claim 16, wherein the base cover includes detents positioned on an upper surface of the base cover, and wherein rotating the pressure applying element and support structure relative to the base cover includes using the detents to determine where to stop rotating the pressure applying element relative to the base cover.

19. The method of claim 16, wherein at least one of the pressure applying element and the base cover is transparent, the method further comprising visualizing the suture threads through the transparent the pressure applying element and the base cover.

20. The method of claim 16, further comprising leaving the vessel clamping pressure device on the wound for a clotting period, and thereafter releasing tension on the suture threads by rotating the spindle to disengage the suture threads.

21. A base cover for a vessel clamping pressure device, comprising:
a bottom having a bottom surface configured to contact skin of a patient at a site of a wound closure;
a base slit extending through the base cover from an outer edge toward a central region, wherein the base slit is configured to receive suture threads through broadside insertion; and
grip wings extending from opposite sides of the base cover, wherein each grip wing has a grip wing contour configured to facilitate gripping by a user.

22. The base cover of claim 21, wherein the bottom surface is curved to conform to contours of the patient's skin.

23. The base cover of claim 21, wherein the base slit includes a base slit inner end that terminates radially outward of a radial center of the base cover.

24. The base cover of claim 21, wherein the base slit has a tapered profile that widens toward the outer edge.

25. The base cover of claim 21, wherein the bottom is manufactured from biocompatible materials that are safe for contact with human skin.

26. The base cover of claim 21, wherein the bottom surface is configured to distribute pressure evenly across a contact area with the patient's skin.

27. The base cover of claim 21, further comprising friction tabs positioned on the bottom surface to prevent slipping on the patient's skin.

28. The base cover of claim 21, wherein the base cover is sufficiently transparent to enable visualization of suture threads and skin beneath the base cover.

29. The base cover of claim 21, wherein the grip wing contour includes textured surfaces configured to enhance grip for a clinician's fingers.

30. The base cover of claim 21, wherein the base slit includes a flared portion at the outer edge providing a wider inlet to facilitate insertion of suture threads.
